(19) United States
(12) Patent Application Publication
(10) Pub. No.: US 2022/0291220 A1
(43) Pub. Date: Sep. 15, 2022

(54) METHODS AND COMPOSITIONS FOR DETECTION AND TREATMENT OF LUNG CANCER

(71) Applicant: OncoCyte Corporation, Alameda, CA (US)

(72) Inventors: Lyndal K. Hesterberg, Alameda, CA (US); Philip R. McQuary, Alameda, CA (US); Karen A.F. Copeland, Alameda, CA (US)

(21) Appl. No.: 17/627,607

(22) PCT Filed: Jul. 15, 2020
(86) PCT No.: PCT/US2020/042146

§ 371 Date: Jan. 14, 2022

Related U.S. Application Data
(60) Provisional application No. 62/874,309, filed on Jul. 15, 2019.

(51) Int. Cl. G01N 33/574; G01N 33/68

(52) U.S. Cl. CPC G01N 33/57423; G01N 33/6872; G01N 2333/475

(57) ABSTRACT

Disclosed herein are methods for detecting whether a subject is at risk of lung cancer based on a blood-based multivariate gene expression classifier. Methods for determining whether to obtain a biopsy in a subject based on a blood-based multivariate gene expression classifier are also disclosed. Further disclosed are methods for treating lung cancer based on a blood-based multivariate gene expression classifier are disclosed. Kits for detecting the expression levels of biomarkers for determining whether a subject is at risk of lung cancer are also disclosed.

### CROSS-REFERENCE TO RELATED APPLICATION

This application claims benefit of U.S. Provisional Application No. 62/874,309, filed Jul. 15, 2019, and is hereby incorporated herein by reference in its entirety.

### FIELD OF THE INVENTION

The present disclosure relates to lung cancer and the detection, diagnosis, and treatment of lung cancer.

### BACKGROUND OF THE INVENTION

Lung cancer is the leading cause of cancer death in the United States. However, lung cancer typically does not cause signs and symptoms in its earliest stages. More than 80 percent of patients with lung cancer are diagnosed at an advanced stage, and only 16.6% of all lung cancer patients are alive five or more years after diagnosis. See Gould M K. Clinical practice. Lung-cancer screening with low-dose computed tomography. N Engl J Med. 2014; 371(19):1813-1820. doi:10.1056/NEJMcp1404071; Howlader N, Noone A M, Krapcho M, Neyman N, Aminou R, Waldron W, Altekruse S F, Kosary C L, Ruhl J, Tatalovich Z, Cho H, Mariotto A, Eisner M P, Lewis D R, Chen H S, Feuer E J, Cronin K A (eds). SEER Cancer Statistics Review, 1975-2009. Therefore, early and accurate detection and diagnosis of lung cancer is important for a patient to achieve the best possible outcome.

Detection and diagnosis of lung cancer typically include two stages: (1) initial evaluation using imaging tests to detect abnormal masses and nodules; and (2) biopsy procedure to determine whether a suspected mass or nodule is benign or malignant.

X-ray imaging or a CT scan can be used in the initial evaluation of lung cancer, with CT scan being more sensitive for revealing smaller lesions in the lung that cannot be detected on an X-ray. One variation of CT scan known as helical low dose computed tomography (LDCT) have shown benefit for high risk patients. However, LDCT screening is not without risk. In the prospective National Lung Cancer Screening Trial (NLST), it was shown that 96.4% of the positive screening results were false positives, leading to unnecessary further testing in those patients. See National Lung Screening Trial Research Team, Aberle D R, Adams A M, et al. Reduced lung-cancer mortality with low-dose computed tomographic screening. N Engl J Med. 2011; 365(5):395-409. doi:10.1056/NEJMoal102873.

Once a mass or nodule is detected in the initial evaluation, tissue samples can be removed in a biopsy procedure and examined for the presence of abnormal cells. A biopsy can be performed in a number of ways including bronchoscopy, mediastinoscopy, and needle biopsy. A biopsy sample can also be taken from lymph nodes or other areas where cancer has spread, such as the liver. Analysis of the biopsy sample identifies whether the suspected mass or nodule is benign or malignant, as well as specific characteristics of the cancer cells present, which helps to determine prognosis and guide treatment.

To reduce unnecessary biopsy testing, noninvasive detection and diagnostic tests are desired to improve the specificity of the initial evaluation, and to provide a definitive diagnosis for benign nodules so that unnecessary invasive procedures could be avoided.

Methods and compositions have been reported for diagnosing lung cancer in a mammalian subject by use of 10 or more selected genes from the blood of the subject. For example, International Publication No. WO 2017/223216 provides 559 target sequences and genes for the diagnosis of lung cancer. However, in practice, use of the listed biomarkers has resulted in a high rate of misdiagnosis for patients with lung nodules in the size range of 0.8 cm-2 cm.

Therefore, improved methods of detecting and diagnosing lung cancer, including new biomarkers that can detect and diagnose lung cancer with improved accuracy are desired. Further, sets of biomarkers that can be used to determine whether lung nodules in the size range of 0.8 cm-2 cm are also desired.

### SUMMARY OF THE INVENTION

The present disclosure provides for, and includes, methods for detecting whether a subject is at risk of lung cancer based on a blood-based multivariate gene expression classifier. The present disclosure further provides for, and includes methods for determining whether to obtain a biopsy in a subject based on a blood-based multivariate gene expression classifier. The present disclosure also provides for, and includes, methods for treating lung cancer based on a blood-based multivariate gene expression classifier. Moreover, the present disclosure provides for, and includes kits for detecting the expression levels of biomarkers for determining whether a subject is at risk of lung cancer.

In an aspect, the present disclosure provides for, and includes a method comprising: determining whether a subject is at risk of lung cancer by: obtaining or having obtained non-invasively a biological sample from the subject; detecting expression level of ABCC13 in the biological sample; and comparing the expression level of ABCC13 to a reference level to determine if the subject is at risk of lung cancer; providing a recommendation that the subject is at risk of lung cancer based on said comparing. In one aspect, the present disclosure provides for, and includes, a method comprising: determining whether a subject is at risk of lung cancer by: obtaining or having obtained non-invasively a biological sample from the subject; detecting expression level of ABCC13 in the biological sample; and comparing the expression level of ABCC13 to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of ABCC13 in the biological sample; comparing the expression level of ABCC13 to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, the present disclosure provides for, and includes, a method comprising: determining whether a subject is at risk of lung cancer by: obtaining or having obtained non-invasively a biological sample from the subject; detecting expression level of ADCK3 in the biological sample; and comparing the expression level of ADCK3 to a reference level to determine if the subject is at risk of lung cancer; providing a recommendation that the subject is at risk of lung cancer based on said comparing. In one aspect, the present disclosure provides for, and includes, a method comprising: determining whether a subject is at risk of lung cancer by: obtaining or having obtained non-invasively a biological sample from the subject; detecting expression level of ADCK3 in the biological sample; and comparing the expression level of ADCK3 to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of ADCK3 in the biological sample; comparing the expression level of ADCK3 to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, the present disclosure provides for, and includes, a method comprising: determining whether a subject is at risk of lung cancer by: obtaining or having obtained non-invasively a biological sample from the subject; detecting expression level of GALNT14 in the biological sample; and comparing the expression level of GALNT14 to a reference level to determine if the subject is at risk of lung cancer; providing a recommendation that the subject is at risk of lung cancer based on said comparing. In an aspect, the present disclosure provides for, and includes, a method comprising: determining whether a subject is at risk of lung cancer by: obtaining or having obtained non-invasively a biological sample from the subject; detecting expression level of GALNT14 in the biological sample; and comparing the expression level of GALNT14 to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of GALNT14 in the biological sample; comparing the expression level of GALNT14 to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, the present disclosure provides for, and includes, a method comprising: determining whether a subject is at risk of lung cancer by: obtaining or having obtained non-invasively a biological sample from the subject; detecting expression level of GNAI2 in the biological sample; and comparing the expression level of GNAI2 to a reference level to determine if the subject is at risk of lung cancer; providing a recommendation that the subject is at risk of lung cancer based on said comparing. In an aspect, the present disclosure provides for, and includes, a method comprising: determining whether a subject is at risk of lung cancer by: obtaining or having obtained non-invasively a biological sample from the subject; detecting expression level of GNAI2 in the biological sample; and comparing the expression level of GNAI2 to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of GNAI2 in the biological sample; comparing the expression level of GNAI2 to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, the present disclosure provides for, and includes, a method comprising: determining whether a subject is at risk of lung cancer by: obtaining or having obtained non-invasively a biological sample from the subject; detecting expression level of PPM1M in the biological sample; and comparing the expression level of PPM1M to a reference level to determine if the subject is at risk of lung cancer; providing a recommendation that the subject is at risk of lung cancer based on said comparing. In an aspect, the present disclosure provides for, and includes, a method comprising: determining whether a subject is at risk of lung cancer by: obtaining or having obtained non-invasively a biological sample from the subject; detecting expression level of PPM1M in the biological sample; and comparing the expression level of PPM1M to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of PPM1M in the biological sample; comparing the expression level of PPM1M to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, the present disclosure provides for, and includes, a method comprising: determining whether a subject is at risk of lung cancer by: obtaining or having obtained non-invasively a biological sample from the subject; detecting expression level of ZER1 in the biological sample; and comparing the expression level of ZER1 to a reference level to determine if the subject is at risk of lung cancer; providing a recommendation that the subject is at risk of lung cancer based on said comparing. In an aspect, the present disclosure provides for, and includes, a method comprising: determining whether a subject is at risk of lung cancer by: obtaining or having obtained non-invasively a biological sample from the subject; detecting expression level of ZER1 in the biological sample; and comparing the expression level of ZER1 to a reference level to determine if the subject is at risk of lung cancer; if the subject is at risk of lung cancer, obtaining or having obtained a biopsy from the subject to test for lung cancer. In an aspect, a method for treating a subject in need of cancer treatment is provided, the method comprising obtaining or having obtained non-invasively a biological sample from the subject; detecting expression levels of biomarkers of ZER1 in the biological sample; comparing the expression level of ZER1 to a reference level to determine if the subject is at risk of lung cancer; and administering or having administered a lung cancer therapy if the subject is determined to be at risk of lung cancer based on the comparing step. In an aspect, the lung cancer therapy is selected from the group consisting of surgery, chemotherapy, radiation therapy, targeted therapy, and immunotherapy. In one aspect, a biological sample is blood.

In an aspect, a method of the present disclosure further comprises detecting expression level of a set of biomarkers comprising one or more biomarkers selected from the list of biomarkers in Table 1, Table 2, and Table 3.

In an aspect, the present disclosure provides for, and includes, a kit comprising a probe for detecting the expression level of ABCC13 for determining whether a subject is at risk of lung cancer. In one aspect, a probe for detecting the expression level of ABCC13 is a nucleic acid probe comprising nucleic acid sequence of SEQ ID NO: 1, complements thereof, or fragments thereof having at least 15 nucleotides.

In an aspect, the present disclosure provides for, and includes, a kit comprising a probe for detecting the expression level of ADCK3 for determining whether a subject is at risk of lung cancer. In one aspect, a probe for detecting the expression level of ADCK3 is a nucleic acid probe comprising nucleic acid sequence of SEQ ID NO: 4, complements thereof, or fragments thereof having at least 15 nucleotides.

In an aspect, the present disclosure provides for, and includes, a kit comprising a probe for detecting the expression level of GALNT14 for determining whether a subject is at risk of lung cancer. In one aspect, a probe for detecting the expression level of GALNT14 is a nucleic acid probe comprising nucleic acid sequence of SEQ ID NO: 22, complements thereof, or fragments thereof having at least 15 nucleotides.

In an aspect, the present disclosure provides for, and includes, a kit comprising a probe for detecting the expression level of GNAI2 for determining whether a subject is at risk of lung cancer. In one aspect, a probe for detecting the expression level of GALNT14 is a nucleic acid probe comprising nucleic acid sequence of SEQ ID NO: 25, complements thereof, or fragments thereof having at least 15 nucleotides.

In an aspect, the present disclosure provides for, and includes, a kit comprising a probe for detecting the expression level of PPM1M for determining whether a subject is at risk of lung cancer. In one aspect, a probe for detecting the expression level of PPM1M is a nucleic acid probe comprising nucleic acid sequence of SEQ ID NO: 91, complements thereof, or fragments thereof having at least 15 nucleotides.

In an aspect, the present disclosure provides for, and includes, a kit comprising a probe for detecting the expression level of ZER1 for determining whether a subject is at risk of lung cancer. In one aspect, a probe for detecting the expression level of ZER1 is a nucleic acid probe comprising nucleic acid sequence of SEQ ID NO: 67, complements thereof, or fragments thereof having at least 15 nucleotides.

In an aspect, a kit of the present disclosure further comprises additional probes for detecting the expression levels of one or more biomarkers selected from the list of biomarkers in Table 1, Table 2, and Table 3.

### BRIEF DESCRIPTION OF THE DRAWINGS

The present disclosure is disclosed with reference to the accompanying drawings, wherein:

FIG. 1A is a plot illustrating the performance of the gene expression classifier in accordance with the present disclosure compared to the Mayo Clinic model;

FIG. 1B is a scatter plot assessing individual classification of all benign and malignant lung nodules in a validation population in accordance with the present disclosure;

FIG. 1C illustrates the calculation of the probability that nodules identified as benign by the gene expression classifier in accordance with the present disclosure truly are benign;

FIG. 2A is a plot of gene expression classifier in accordance with the present disclosure in distinguishing benign from malignant nodules across gender;

FIG. 2B is a plot of gene expression classifier in accordance with the present disclosure in distinguishing benign from malignant nodules across various nodule sizes;

FIG. 2C is a plot of gene expression classifier in accordance with the present disclosure in distinguishing benign from malignant nodules across various fungal geographic distribution;

FIG. 3 is a plot of gene expression classifier performance for clinically challenging cases in accordance with the present disclosure.

### DETAILED DESCRIPTION

In an aspect, the present disclosure provides for, and includes, a multivariate gene expression classifier that identifies benign from malignant nodules between 0.5 cm to 3.0 cm with high accuracy in a diverse population of current and former smokers. In one aspect, the classifier of the present description, which is based on biomarkers only, significantly outperforms the Mayo Model for cancer risk that utilizes nodule size as one of the variables. In an aspect, the classifier of the present description is a non-invasive gene expression classifier that provides a biological assessment of the risk of cancer with predictive value independent of nodule size or clinical factors.

The present disclosure provides for, and includes, methods for detecting whether a subject is at risk of lung cancer based on a blood-based multivariate gene expression classifier. The present disclosure further provides for, and includes methods for determining whether to obtain a biopsy in a subject based on a blood-based multivariate gene expression classifier. The present disclosure also provides for, and includes, methods for treating lung cancer based on a blood-based multivariate gene expression classifier. Moreover, the present disclosure provides for, and includes kits for detecting the expression levels of biomarkers for determining whether a subject is at risk of lung cancer.

In an aspect, methods in accordance with the present disclosure support improved care for patients for whom clinical management is not straightforward by identifying the subset of patients at low risk of malignancy. In an aspect, methods of the present disclosure support improved care for patients having pulmonary nodules between 0.5 cm to 3.0 cm, such as, between 0.8 cm to 3.0 cm, or between 0.8 cm to 2.0 cm. In an aspect, malignant nodules are identified by the Mayo Clinic model. See Swensen S J, Silverstein M D, Ilstrup D M, Schleck C D, Edell E S. The probability of malignancy in solitary pulmonary nodules. Application to small radiologically indeterminate nodules. Arch Intern Med. 1997; 157(8):849-855. In one aspect malignant nodules are identified by predictive models selected from the group consisting of the Veterans Association model (Gould M K, Ananth L, Barnett P G. A clinical model to estimate the pretest probability of lung cancer in patients with solitary pulmonary nodules. Chest. 2007; 131:383-8), the Brock University model (McWilliams A, Tammemagi M C, Mayo J R, Roberts H, Liu G, Soghrati K, et al. Probability of cancer in pulmonary nodules detected on first screening CT. N Engl J Med. 2013; 369:910-9), and the Herder Model (Herder G J, van Tinteren H, Golding R P, Kostense P J, Comans E F, Smit E F, et al. Clinical prediction model to characterize pulmonary nodules: validation and added value of 18F-fluorodeoxyglucose positron emission tomography. Chest. 2005; 128:2490-6.)

In an aspect, methods and compositions of the present disclosure detect, diagnose, or treat lung cancer. In one aspect, lung cancer is non-small cell lung cancer (NSCLC). In an aspect, lung cancer is lung adenocarcinoma (AC or LAC). In one aspect, lung cancer is lung squamous cell carcinoma (SCC or LSCC). In an aspect, lung cancer is a stage I or stage II NSCLC. In one aspect, lung cancer is a mixture of early and late stages and types of NSCLC. In an aspect, lung cancer is a lung carcinoid tumor. In an aspect, a lung carcinoid tumor is a typical carcinoid tumor of the lung. In one aspect, a lung carcinoid tumor is an atypical carcinoid tumor of the lung. In an aspect, lung cancer is adenosquamous carcinoma of the lung (ASC), which is a malignancy containing components of AC and SCC.

Unless defined otherwise herein, terms are to be understood according to conventional usage by those of ordinary skill in the relevant art. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

All publications, patents, and patent applications mentioned in this disclosure are herein incorporated by reference to the same extent as if each individual publication, patent, or patent application was specifically and individually indicated to be incorporated by reference.

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination, or A, B, and C in combination.

As used herein, terms in the singular and the singular forms “a,” “an,” and “the,” for example, include plural referents unless the content clearly dictates otherwise.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure. Whenever the phrase “comprising” is used, variations such as “consisting essentially of” and “consisting of” are also contemplated.

The term “about,” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

As used herein, a “subject” can be a human or an animal.

As used herein, the terms “administer,” “administering,” or “administration” in reference to a dosage form of the disclosure refers to the act of introducing the dosage form into the system of a subject in need of treatment. When a dosage form of the disclosure is given in combination with one or more other active agents (in their respective dosage forms), “administration” and its variants are each understood to include concurrent and/or sequential introduction of the dosage form and the other active agents. Administration of any of the described dosage forms includes simultaneous (parallel) administration, sequential (stepwise) administration, co-administration, or separate administration, in which the therapies are administered separately at approximately the same time, e.g., within about a few seconds to a few hours of one another.

As used herein, the term “biomarker” refers to a biological molecule found in blood, other body fluids, or tissues that is a sign of a normal or abnormal process, or of a condition or disease, such as cancer. Biomarkers typically differentiate an affected patient from a person without the disease, due to a number of factors including germline or somatic mutations, transcriptional changes, and post-translational modifications. A biomarker can be selected from a group consisting of a protein (e.g., an enzyme or receptor), a nucleic acid (e.g., a messenger RNA, a microRNA or other non-coding RNA), an antibody, and a peptide. In an aspect, a biomarker can also be a collection of alterations, such as gene expression, proteomic, and metabolomic signatures.

As used herein, the term “gene expression classifier” refers to a set of biomarkers for which the gene expression profile is used to diagnose lung cancer.

As used herein, the terms “control” and “control subject” refer to the source of a reference level of a particular gene, reference gene expression profiles, as well as particular panel of control subjects described herein.

As used herein, the term “sample” means any biological fluid or tissue that contains immune cells, cancer cells, or a combination thereof.

As used herein, the term “cancer” refers to or describes the physiological condition in mammals that is typically characterized by unregulated cell growth.

As used herein, the term “tumor” refers to all neoplastic cell growth and proliferation, whether malignant or benign, and all pre-cancerous and cancerous cells and tissues.

As used herein, the term “nodule” refers to an abnormal buildup of tissue which is benign.

As used herein, the term “cancerous tumor” refers to a malignant tumor.

As used herein, the term “malignant” refers to a disease state where abnormal cells divide without control and can invade nearby tissues.

As used herein, the terms “benign” or “nonmalignant” refer to a generally harmless state where cells can grow larger but do not spread to other parts of the body.

As used herein, the terms “diagnosis” and “evaluation” refer to a diagnosis of a lung cancer, a diagnosis of a stage of lung cancer, a diagnosis of a type or classification of a lung cancer, a diagnosis or detection of a recurrence of a lung cancer, a diagnosis or detection of a regression of a lung cancer, a prognosis of a lung cancer, or an evaluation of the response of a lung cancer to a surgical or non-surgical therapy. In an aspect, the terms “diagnosis” and “evaluation” refer to distinguishing between a cancerous tumor and a benign nodule.

As used herein, the term “differential expression” refers to an upregulation of one or more selected genes in comparison to the reference or control; a downregulation of one or more selected genes in comparison to the reference or control; or a combination of certain upregulated genes and down regulated genes.

As used herein, the term “microarray” refers to an ordered arrangement of hybridizable array elements, such as polynucleotide, oligonucleotide, or polypeptide probes, on a substrate.

As used herein, the term “polynucleotide,” whether used in singular or plural form, generally refers to any polyribonucleotide or polydeoxyribonucleotide, which can be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that can be single-stranded or, more typically, double-stranded or include single- and double-stranded regions. In an aspect, the term “polynucleotide” refers to triple-stranded regions comprising RNA or DNA or both RNA and DNA. The strands in such regions can be from the same molecule or from different molecules. The regions can include all of one or more of the molecules, but more typically involve only a region of some of the molecules. One of the molecules of a triple-helical region often is an oligonucleotide. In an aspect, the term “polynucleotide” specifically includes cDNAs. In an aspect, the term “polynucleotide” includes DNAs (including cDNAs) and RNAs that contain one or more modified bases. DNA molecules comprise the nucleotide bases adenine (A), guanine (G), thymine (T), cytosine (C). Uracil (U) replaces thymine in RNA molecules. The symbol “N” can be used to represent any nucleotide base (e.g., A, G, C, T, or U). In an aspect, DNAs or RNAs with backbones modified for stability or for other reasons are “polynucleotides” as that term is intended herein. In an aspect, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

As used herein, the term “oligonucleotide” refers to a relatively short polynucleotide, including, without limitation, single-stranded deoxyribonucleotides, single- or double-stranded ribonucleotides, RNA:DNA hybrids and double-stranded DNAs. In an aspect, oligonucleotides, such as single-stranded DNA probe oligonucleotides, are synthesized by chemical methods, for example using automated oligonucleotide synthesizers that are commercially available. In an aspect, oligonucleotides is made by a variety of other methods, including in vitro recombinant DNA-mediated techniques and by expression of DNAs in cells and organisms.

As used herein, the terms “polypeptide”, “peptide” and “protein” are used interchangeably to refer to a polymer of amino acid residues. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

As used herein, “encoding” refers to a polynucleotide encoding for the amino acids of a polypeptide or a non-coding RNA molecule. A series of three nucleotide bases encodes one amino acid.

As used herein, the terms “differentially expressed gene,” “differential gene expression” and their synonyms, which are used interchangeably, refer to a gene whose expression is activated to a higher or lower level in a subject suffering from a disease, specifically cancer, such as lung cancer, relative to its expression in a control subject, such as a subject having a benign nodule. In an aspect, the terms also include genes whose expression is activated to a higher or lower level at different stages of the same disease. It is also understood that a differentially expressed gene can be either activated or inhibited at the nucleic acid level or protein level, or can be subject to alternative splicing to result in a different polypeptide product. Such differences can be evidenced by a change in mRNA levels, surface expression, secretion or other partitioning of a polypeptide, for example.

As used herein, the term “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are deemed to have “sequence similarity” or “similarity.”

Detection of Biomarkers

In an aspect, a biological sample obtained from a subject is whole blood. In one aspect, a biological sample is selected from the group consisting of peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. In an aspect, a biological sample can be diluted with saline, buffer, or a physiologically acceptable diluent. In one aspect, a biological sample can be concentrated by lyophilization, vacuum evaporation, or nitrogen blow-down evaporation.

In an aspect, biomarkers of the present disclosure can be detected in the circulation (whole blood, serum, or plasma) or excretions or secretions (stool, urine, sputum, or nipple discharge), and thus easily assessed non-invasively and serially, or can be tissue-derived, and require either biopsy or special imaging for evaluation. In an aspect, biomarkers are genetic biomarkers that can be inherited. In an aspect, genetic biomarkers are detected as sequence variations in germ line DNA isolated from whole blood, sputum, or buccal cells, or can be somatic, and identified as mutations in DNA derived from tumor tissue.

In an aspect, a biomarker of the present disclosure is selected from the biomarkers in Table 1.

TABLE 1
Biomarkers used in a gene expression classifier for lung cancer

| | | | Full Length | Full Length |
|---|---|---|---|---|
| | | Biomarker | Nucleic Acid | Polypeptide |
| Biomarker | Description | SEQ ID NO | SEQ ID NO | SEQ ID NO |
| ABCC13 | ATP-binding cassette | 1 | 2 | 3 |
| | transporters | | | |
| ADCK3 | an atypical kinase thought to | 4 | 5 | 6 |
| | play a regulatory role in | | | |
| | coenzyme Q10 biosynthesis | | | |
| AMD1 | adenosylimethionine | 7 | 8 | 9 |
| | decarboxylase 1 | | | |
| CIR1 | Corepressor interacting with | 10 | 11 | 12 |
| | RBPJ 1 | | | |
| CLK3 | serine/threonine type protein | 13 | 14 | 15 |
| | kinase with a non-conserved N- | | | |
| | terminal domain | | | |
| DNAJB1 | DnaJ homolog subfamily B | 16 | 17 | 18 |
| | member 1 | | | |
| EPHX2 | soluble epoxide hydrolase | 19 | 20 | 21 |
| GALNT14 | polypeptide N- | 22 | 23 | 24 |
| | acetylgalactosaminyltransferase 14 | | | |
| GNAI2 | guanine nucleotide-binding | 25 | 26 | 27 |
| | protein G(i), alpha-2 subunit | | | |
| GSR | glutathione reductase, also | 28 | 29 | 30 |
| | known as glutathione-disulfide | | | |
| | reductase | | | |
| IFI27 | interferon alpha-inducible | 31 | 32 | 33 |
| | protein 27 | | | |
| INO80C | INO80 complex subunit C | 34 | 35 | 36 |
| LGR6 | leucine-rich repeat-containing | 37 | 38 | 39 |
| | G-protein coupled receptor 6 | | | |
| PDCD6IP | programmed cell death 6- | 40 | 41 | 42 |
| | interacting protein | | | |
| PSMB4 | 20S proteasome subunit beta-7 | 43 | 44 | 45 |
| | protein | | | |
| SCAF4 | arginine/serine-rich splicing | 46 | 47 | 48 |
| | factor family protein. | | | |
| SNRPD3 | small nuclear ribonucleoprotein | 49 | 50 | 51 |
| | Sm D3 | | | |
| STAMBP | STAM-binding protein | 52 | 53 | 54 |
| TMEM8A | Transmembrane protein 8A | 55 | 56 | 57 |
| UBAP2 | Ubiquitin-associated protein 2 | 58 | 59 | 60 |
| VNN1 | Pantetheinase | 61 | 62 | 63 |
| VPS29 | vacuolar protein sorting protein | 64 | 65 | 66 |
| ZER1 | a subunit of an E3 ubiquitin | 67 | 68 | 69 |
| | ligase complex | | | |

In an aspect, a gene expression classifier, or a gene panel, comprises all 23 biomarkers in Table 1. In an aspect, a gene expression classifier comprises a biomarker selected from the biomarkers in Table 1. In an aspect, a gene expression classifier comprises at least 5, at least 10, at least 15, or at least 20 biomarkers selected from the biomarkers in Table 1.

In an aspect, a gene expression classifier comprises ABCC13. In an aspect, a gene expression classifier comprises ABCC13 and at least 1, at least 5, at least 10, at least 15, or at least 20 biomarkers selected from the biomarkers in Table 1.

In an aspect, a gene expression classifier comprises ADCK3. In an aspect, a gene expression classifier comprises ADCK3 and at least 1, at least 5, at least 10, at least 15, or at least 20 biomarkers selected from the biomarkers in Table 1.

In an aspect, a gene expression classifier comprises GALNT14. In an aspect, a gene expression classifier comprises GALNT14 and at least 1, at least 5, at least 10, at least 15, or at least 20 biomarkers selected from the biomarkers in Table 1.

In an aspect, a gene expression classifier comprises GNAI2. In an aspect, a gene expression classifier comprises GNAI2 and at least 1, at least 5, at least 10, at least 15, or at least 20 biomarkers selected from the biomarkers in Table 1.

In an aspect, a gene expression classifier comprises ZER1. In an aspect, a gene expression classifier comprises ZER1 and at least 1, at least 5, at least 10, at least 15, or at least 20 biomarkers selected from the biomarkers in Table 1.

In an aspect, a biomarker of the present disclosure is selected from the biomarkers in Table 1 and Table 2.

TABLE 2
Additional biomarkers used in a gene expression classifier for lung cancer

| | | | Full Length | Full Length |
|---|---|---|---|---|
| | | Biomarker | Nucleic Acid | Polypeptide |
| Biomarker | Description | SEQ ID NO | SEQ ID NO | SEQ ID NO |
| ANKHD1 | Ankyrin Repeat And KH | 70 | 71 | 72 |
| | Domain-Containing Protein 1 | | | |
| CLCN4 | Chloride Voltage-Gated | 73 | 74 | 75 |
| | Channel 4 | | | |
| CTSH | Cathepsin H | 76 | 77 | 78 |
| HDAC3 | Histone Deacetylase 3 | 79 | 80 | 81 |
| ITPR1 | Inositol 1,4,5-Trisphosphate | 82 | 83 | 84 |
| | Receptor Type 1 | | | |
| LILRA5 | Leukocyte Immunoglobulin | 85 | 86 | 87 |
| | Like Receptor A5 | | | |
| PNPLA6 | Patatin-Like Phospholipase | 88 | 89 | 90 |
| | Domain-Containing Protein 6 | | | |
| PPM1M | Protein Phosphatase, | 91 | 92 | 93 |
| | Mg2+/Mn2+ Dependent 1M | | | |
| PUM1 | Pumilio RNA Binding Family | 94 | 95 | 96 |
| | Member 1 | | | |
| RBPMS2 | RNA Binding Protein, MRNA | 97 | 98 | 99 |
| | Processing Factor 2 | | | |
| SLC25A20 | Solute Carrier Family 25 | 100 | 101 | 102 |
| | Member 20 | | | |
| TPR | Translocated Promoter Region, | 103 | 104 | 105 |
| | Nuclear Basket Protein | | | |
| WDFY3 | WD Repeat And FYVE | 106 | 107 | 108 |
| | Domain Containing Protein 3 | | | |

In an aspect, a gene expression classifier, or a gene panel, comprises all 13 biomarkers in Table 2. In an aspect, a gene expression classifier comprises all 36 biomarkers in Table 1 and Table 2. In an aspect, a gene expression classifier comprises a biomarker selected from the biomarkers in Table 2. In an aspect, a gene expression classifier comprises at least 5 or at least 10 biomarkers selected from the biomarkers in Table 2. In an aspect, a gene expression classifier comprises at least 5, at least 10, at least 15, at least 20, at least 25, at least 30, or at least 35 biomarkers selected from the 36 biomarkers in Table 1 and Table 2.

In an aspect, a gene expression classifier comprises ABCC13. In an aspect, a gene expression classifier comprises ABCC13 and at least 1, at least 5, at least 10, at least 15, at least 20, at least 25, at least 30, or at least 35 biomarkers selected from the 36 biomarkers in Table 1 and Table 2. In an aspect, a gene expression classifier comprises ABCC13 and a biomarker selected from the group consisting of ADCK3, GALNT14, GNAI2, PPM1M, and ZER1. In an aspect, a gene expression classifier comprises ABCC13 and two biomarkers selected from the group consisting of ADCK3, GALNT14, GNAI2, PPM1M, and ZER1. In an aspect, a gene expression classifier comprises ABCC13 and three biomarkers selected from the group consisting of ADCK3, GALNT14, GNAI2, PPM1M, and ZER1. In an aspect, a gene expression classifier comprises ABCC13 and four biomarkers selected from the group consisting of ADCK3, GALNT14, GNAI2, PPM1M, and ZER1.

In an aspect, a gene expression classifier comprises ADCK3. In an aspect, a gene expression classifier comprises ADCK3 and at least 1, at least 5, at least 10, at least 15, at least 20, at least 25, at least 30, or at least 35 biomarkers selected from the 36 biomarkers in Table 1 and Table 2. In an aspect, a gene expression classifier comprises ADCK3 and a biomarker selected from the group consisting of ABCC13, GALNT14, GNAI2, PPM1M, and ZER1. In an aspect, a gene expression classifier comprises ADCK3 and two biomarkers selected from the group consisting of ABCC13, GALNT14, GNAI2, PPM1M, and ZER1. In an aspect, a gene expression classifier comprises ADCK3 and three biomarkers selected from the group consisting of ABCC13, GALNT14, GNAI2, PPM1M, and ZER1. In an aspect, a gene expression classifier comprises ADCK3 and four biomarkers selected from the group consisting of ABCC13, GALNT14, GNAI2, PPM1M, and ZER1.

In an aspect, a gene expression classifier comprises GALNT14. In an aspect, a gene expression classifier comprises GALNT14 and at least 1, at least 5, at least 10, at least 15, at least 20, at least 25, at least 30, or at least 35 biomarkers selected from the 36 biomarkers in Table 1 and Table 2. In an aspect, a gene expression classifier comprises GALNT14 and a biomarker selected from the group consisting of ABCC13, ADCK3, GNAI2, PPM1M, and ZER1. In an aspect, a gene expression classifier comprises GALNT14 and two biomarkers selected from the group consisting of ABCC13, ADCK3, GNAI2, PPM1M, and ZER1. In an aspect, a gene expression classifier comprises GALNT14 and three biomarkers selected from the group consisting of ABCC13, ADCK3, GNAI2, PPM1M, and ZER1. In an aspect, a gene expression classifier comprises GALNT14 and four biomarkers selected from the group consisting of ABCC13, ADCK3, GNAI2, PPM1M, and ZER1.

In an aspect, a gene expression classifier comprises GNAI2. In an aspect, a gene expression classifier comprises GNAI2 and at least 1, at least 5, at least 10, at least 15, at least 20, at least 25, at least 30, or at least 35 biomarkers selected from the 36 biomarkers in Table 1 and Table 2. In an aspect, a gene expression classifier comprises GNAI2 and a biomarker selected from the group consisting of ABCC13, ADCK3, GALNT14, PPM1M, and ZER1. In an aspect, a gene expression classifier comprises GNAI2 and two biomarkers selected from the group consisting of ABCC13, ADCK3, GALNT14, PPM1M, and ZER1. In an aspect, a gene expression classifier comprises GNAI2 and three biomarkers selected from the group consisting of ABCC13, ADCK3, GALNT14, PPM1M, and ZER1. In an aspect, a gene expression classifier comprises GNAI2 and four biomarkers selected from the group consisting of ABCC13, ADCK3, GALNT14, PPM1M, and ZER1.

In an aspect, a gene expression classifier comprises PPM1M. In an aspect, a gene expression classifier comprises PPM1M and at least 1, at least 5, at least 10, at least 15, at least 20, at least 25, at least 30, or at least 35 biomarkers selected from the 36 biomarkers in Table 1 and Table 2. In an aspect, a gene expression classifier comprises PPM1M and a biomarker selected from the group consisting of ABCC13, ADCK3, GALNT14, GNAI2, and ZER1. In an aspect, a gene expression classifier comprises PPM1M and two biomarkers selected from the group consisting of ABCC13, ADCK3, GALNT14, GNAI2, and ZER1. In an aspect, a gene expression classifier comprises PPM1M and three biomarkers selected from the group consisting of ABCC13, ADCK3, GALNT14, GNAI2, and ZER1. In an aspect, a gene expression classifier comprises PPM1M and four biomarkers selected from the group consisting of ABCC13, ADCK3, GALNT14, GNAI2, and ZER1.

In an aspect, a gene expression classifier comprises ZER1. In an aspect, a gene expression classifier comprises ZER1 and at least 1, at least 5, at least 10, at least 15, at least 20, at least 25, at least 30, or at least 35 biomarkers selected from the 36 biomarkers in Table 1 and Table 2. In an aspect, a gene expression classifier comprises ZER1 and a biomarker selected from the group consisting of ABCC13, ADCK3, GALNT14, GNAI2, and PPM1M. In an aspect, a gene expression classifier comprises ZER1 and two biomarkers selected from the group consisting of ABCC13, ADCK3, GALNT14, GNAI2, and PPM1M. In an aspect, a gene expression classifier comprises ZER1 and three biomarkers selected from the group consisting of ABCC13, ADCK3, GALNT14, GNAI2, and PPM1M. In an aspect, a gene expression classifier comprises ZER1 and four biomarkers selected from the group consisting of ABCC13, ADCK3, GALNT14, GNAI2, and PPM1M.

In an aspect, a gene expression classifier comprises one or more biomarkers selected from the group consisting of ABCC13, ADCK3, GALNT14, GNAI2, PPM1M, and ZER1; and one or more biomarkers selected from the group consisting of AMD1, CLCN4, PDCD6IP, PUM1, RBPMS2, and UBAP2.

In an aspect, a biomarker of the present disclosure is selected from the biomarkers in Table 1, Table 2, and Table 3.

TABLE 3
Additional biomarkers used in a gene expression classifier for lung cancer

| | | | Full Length | Full Length |
|---|---|---|---|---|
| | | Biomarker | Nucleic Acid | Polypeptide |
| Biomarker | Description | SEQ ID NO | SEQ ID NO | SEQ ID NO |
| ACHE | Acetylcholinesterase | 109 | 110 | 111 |
| | (Cartwright Blood Group) | | | |
| ACP1 | Acid Phosphatase 1 | 112 | 113 | 114 |
| AHCYL1 | Inositol 1,4,5-Trisphosphate | 115 | 116 | 117 |
| | Receptor-Binding Protein | | | |
| AKR1C1 | Aldo-Keto Reductase Family 1 | 118 | 119 | 120 |
| | Member C1 | | | |
| APOL1 | Apolipoprotein L1 | 121 | 122 | 123 |
| APOL3 | Apolipoprotein L3 | 124 | 125 | 126 |
| ARCN1 | Coatomer Protein Complex, | 127 | 128 | 129 |
| | Subunit Delta | | | |
| ARHGAP25 | Rho GTPase Activating | 130 | 131 | 132 |
| | Protein 25 | | | |
| ARHGEF3 | Rho Guanine Nucleotide | 133 | 134 | 135 |
| | Exchange Factor 3 | | | |
| ARPC2 | Actin Related Protein 2/3 | 136 | 137 | 138 |
| | Complex Subunit 2 | | | |
| ATXN2L | Ataxin-2-Like Protein | 139 | 140 | 141 |
| BCAT1 | Branched Chain Amino Acid | 142 | 143 | 144 |
| | Transaminase 1 | | | |
| BCL9L | B-Cell Lymphoma 9-Like | 145 | 146 | 147 |
| | Protein | | | |
| BTG1 | BTG Anti-Proliferation Factor | 148 | 149 | 150 |
| | 1 | | | |
| C1orf123/ | Chromosome 1 Open Reading | 151 | 152 | 153 |
| CZIB | Frame 23 | | | |
| C1orf43 | Chromosome 1 Open Reading | 154 | 155 | 156 |
| | Frame 43 | | | |
| CD177 | CD177 Molecule | 157 | 158 | 159 |
| CD300LD | CD300 Molecule Like Family | 160 | 161 | 162 |
| | Member D | | | |
| CDC42EP2 | CDC42 Effector Protein 2 | 163 | 164 | 165 |
| CEACAM3 | Carcinoembryonic Antigen | 166 | 167 | 168 |
| | Related Cell Adhesion | | | |
| | Molecule 3 | | | |
| CHMP2A | Charged Multivesicular Body | 169 | 170 | 171 |
| | Protein 2A | | | |
| CHST11 | Carbohydrate Sulfotransferase | 172 | 173 | 174 |
| | 11 | | | |
| CMBL | Carboxymethylenebutenolidase | 175 | 176 | 177 |
| | Homolog | | | |
| CRAT | Carnitine O-Acetyltransferase | 178 | 179 | 180 |
| CRIP2 | Cysteine Rich Protein 2 | 181 | 182 | 183 |
| CXCL1 | C—X—C Motif Chemokine | 184 | 185 | 186 |
| | Ligand 1 | | | |
| CXCR2P1 | C—X—C Motif Chemokine | 187 | 188 | non-coding |
| | Receptor 2 Pseudogene 1 | | | |
| DCTN4 | Dynactin Subunit 4 | 190 | 191 | 192 |
| DDX11L2 | DEAD/H-Box Helicase 11 | 193 | 194 | 195 |
| | Like 2 | | | |
| DDX11L9 | DEAD/H-Box Helicase 11 | 196 | 197 | non-coding |
| | Like 9 | | | |
| DHRS4 | Dehydrogenase/Reductase 4 | 199 | 200 | 201 |
| DHX16 | DEAH-Box Helicase 16 | 202 | 203 | 204 |
| DHX8 | DEAH-Box Helicase 8 | 205 | 206 | 207 |
| EDAR | Ectodysplasin A Receptor | 208 | 209 | 210 |
| EIF1AY | Eukaryotic Translation | 211 | 212 | 213 |
| | Initiation Factor 1A Y-Linked | | | |
| EIF2B1 | Eukaryotic Translation | 214 | 215 | 216 |
| | Initiation Factor 2B Subunit | | | |
| | Alpha | | | |
| EIF3C | Eukaryotic Translation | 217 | 218 | 219 |
| | Initiation Factor 3 Subunit C | | | |
| ELF3 | E74 Like ETS Transcription | 220 | 221 | 222 |
| | Factor 3 | | | |
| EMC7 | ER Membrane Protein | 223 | 224 | 225 |
| | Complex Subunit 7 | | | |
| EWSR1 | EWS RNA Binding Protein 1 | 226 | 227 | 228 |
| FAIM3/ | Fas Apoptotic Inhibitory | 229 | 230 | 231 |
| FCMR | Molecule 3 | | | |
| FAM105B/ | Ubiquitin Thioesterase Otulin | 232 | 233 | 234 |
| OTULIN | | | | |
| FAM193A | Family With Sequence | 235 | 236 | 237 |
| | Similarity 193 Member A | | | |
| FAM26F/ | Calcium Homeostasis | 238 | 239 | 240 |
| CALHM6 | Modulator Family Member 6 | | | |
| FAM86HP | Family With Sequence | 241 | 242 | non-coding |
| | Similarity 86 Member H, | | | |
| | Pseudogene | | | |
| FAS | Fas Cell Surface Death | 244 | 245 | 246 |
| | Receptor | | | |
| FBXW2 | F-Box And WD-40 Domain- | 247 | 248 | 249 |
| | Containing Protein 2 | | | |
| FCRL6 | Fc Receptor-Like Protein 6 | 250 | 251 | 252 |
| FGFBP2 | Fibroblast Growth Factor | 253 | 254 | 255 |
| | Binding Protein 2 | | | |
| FMNL1 | Formin-Like Protein 1 | 256 | 257 | 258 |
| FMNL3 | Formin-Like Protein 3 | 259 | 260 | 261 |
| FRG1B/ | FSHD Region Gene 1 Family | 262 | 263 | non-coding |
| FRG1BP | Member B, Pseudogene | | | |
| G0S2 | G0/G1 Switch 2 | 265 | 266 | 267 |
| GADD45A | Growth Arrest And DNA | 268 | 269 | 270 |
| | Damage Inducible Alpha | | | |
| GNA12 | G Protein Subunit Alpha 12 | 271 | 272 | 273 |
| GPR56 | Adhesion G Protein-Coupled | 274 | 275 | 276 |
| | Receptor G1 | | | |
| TPX2 | Microtubule Nucleation Factor | 277 | 278 | 279 |
| GUSBP5 | GUSB Pseudogene 5 | 280 | 281 | non-coding |
| GZMB | Granzyme B | 283 | 284 | 285 |
| GZMH | Granzyme H | 286 | 287 | 288 |
| HEMGN | Hemogen | 289 | 290 | 291 |
| HIST1H3D | Histone Cluster 1 H3 Family | 292 | 293 | 294 |
| | Member D | | | |
| HMBS | Hydroxymethylbilane Synthase | 295 | 296 | 297 |
| HP | Haptoglobin | 298 | 299 | 300 |
| HSPA6 | Heat Shock Protein Family A | 301 | 302 | 303 |
| | (Hsp70) Member 6 | | | |
| HSPA7 | Heat Shock Protein Family A | 304 | 305 | non-coding |
| | (Hsp70) Member 7 | | | |
| IL9R | Interleukin 9 Receptor | 307 | 308 | 309 |
| INSL3 | Leydig Insulin-Like Peptide | 310 | 311 | 312 |
| IRAK3 | Interleukin 1 Receptor | 313 | 314 | 315 |
| | Associated Kinase 3 | | | |
| ITGB3 | Integrin Subunit Beta 3 | 316 | 317 | 318 |
| KCNJ15 | Potassium Voltage-Gated | 319 | 320 | 321 |
| | Channel Subfamily J Member | | | |
| | 15 | | | |
| KDM5D | Lysine Demethylase 5D | 322 | 323 | 324 |
| KIAA0317/ | Apoptosis Resistant E3 | 325 | 326 | 327 |
| AREL1 | Ubiquitin Protein Ligase 1 | | | |
| LAP3 | Leucine Aminopeptidase 3 | 328 | 329 | 330 |
| LPAR1 | Lysophosphatidic Acid | 331 | 332 | 333 |
| | Receptor 1 | | | |
| LRBA | LPS Responsive Beige-Like | 334 | 335 | 336 |
| | Anchor Protein | | | |
| LRP1 | LDL Receptor Related Protein | 337 | 338 | 339 |
| | 1 | | | |
| MATK | Megakaryocyte-Associated | 340 | 341 | 342 |
| | Tyrosine Kinase | | | |
| MAX | MYC Associated Factor X | 343 | 344 | 345 |
| MPL | MPL Proto-Oncogene, | 346 | 347 | 348 |
| | Thrombopoietin Receptor | | | |
| MTCH1 | Mitochondrial Carrier 1 | 349 | 350 | 351 |
| NAIP | NLR Family Apoptosis | 352 | 353 | 354 |
| | Inhibitory Protein | | | |
| ORM1 | Orosomucoid 1 | 355 | 356 | 357 |
| ORM2 | Orosomucoid 2 | 358 | 359 | 360 |
| PDE3A | Phosphodiesterase 3A | 361 | 362 | 363 |
| PI3 | Peptidase Inhibitor 3 | 364 | 365 | 366 |
| PLK1 | Polo Like Kinase 1 | 367 | 368 | 369 |
| PPP1CB | Protein Phosphatase 1 | 370 | 371 | 372 |
| | Catalytic Subunit Beta | | | |
| PPP1R13L | Protein Phosphatase 1 | 373 | 374 | 375 |
| | Regulatory Subunit 13 Like | | | |
| PRKY | Protein Kinase Y-Linked | 376 | 377 | non-coding |
| | (Pseudogene) | | | |
| PRR12 | Proline Rich 12 | 379 | 380 | 381 |
| PSMB2 | Proteasome Subunit Beta 2 | 382 | 383 | 384 |
| PSMC1 | Proteasome 26S Subunit, | 385 | 386 | 387 |
| | ATPase 1 | | | |
| PTGS2 | Prostaglandin-Endoperoxide | 388 | 389 | 390 |
| | Synthase 2 | | | |
| QPCT | Glutaminyl-Peptide | 391 | 392 | 393 |
| | Cyclotransferase | | | |
| RAB7A | RAB7A, Member RAS | 394 | 395 | 396 |
| | Oncogene Family | | | |
| REEP5 | Receptor Accessory Protein 5 | 397 | 398 | 399 |
| RNF11 | Ring Finger Protein 11 | 400 | 401 | 402 |
| RNF114 | Ring Finger Protein 114 | 403 | 404 | 405 |
| RNF14 | Ring Finger Protein 14 | 406 | 407 | 408 |
| RPS4Y1 | Ribosomal Protein S4 Y- | 409 | 410 | 411 |
| | Linked 1 | | | |
| RRBP1 | Ribosome Binding Protein 1 | 412 | 413 | 414 |
| RRN3P3 | RRN3 Homolog, RNA | 415 | 416 | non-coding |
| | Polymerase 1 Transcription | | | |
| | Factor Pseudogene 3 | | | |
| S100B | S100 Calcium Binding Protein | 418 | 419 | 420 |
| | B | | | |
| SAMD3 | Sterile Alpha Motif Domain- | 421 | 422 | 423 |
| | Containing Protein 3 | | | |
| SATB1 | SATB Homeobox 1 | 424 | 425 | 426 |
| SCGB3A1 | Secretoglobin Family 3A | 427 | 428 | 429 |
| | Member 1 | | | |
| SLC14A1 | Solute Carrier Family 14 | 430 | 431 | 432 |
| | Member 1 (Kidd Blood Group) | | | |
| SLC22A18AS | Solute Carrier Family 22 | 433 | 434 | 435 |
| | Member 18 Antisense | | | |
| SLC6A8 | Solute Carrier Family 6 | 436 | 437 | 438 |
| | Member 8 | | | |
| SMARCC1 | SWI/SNF Related, Matrix | 439 | 440 | 441 |
| | Associated, Actin Dependent | | | |
| | Regulator Of Chromatin | | | |
| | Subfamily C Member 1 | | | |
| SPDYC | Speedy/RINGO Cell Cycle | 442 | 443 | 444 |
| | Regulator Family Member C | | | |
| SSBP3 | Single Stranded DNA Binding | 445 | 446 | 447 |
| | Protein 3 | | | |
| ST6GAL1 | ST6 Beta-Galactoside Alpha- | 448 | 449 | 450 |
| | 2,6-Sialyltransferase 1 | | | |
| STK17B | Serine/Threonine Kinase 17b | 451 | 452 | 453 |
| SUPT5H | SPT5 Homolog, DSIF | 454 | 455 | 456 |
| | Elongation Factor Subunit | | | |
| TAS2R43 | Taste 2 Receptor Member 43 | 457 | 458 | 459 |
| TBP | TATA-Box Binding Protein | 460 | 461 | 462 |
| TDRD9 | Tudor Domain Containing 9 | 463 | 464 | 465 |
| TGFBR3 | Transforming Growth Factor | 466 | 467 | 468 |
| | Beta Receptor 3 | | | |
| THBS1 | Thrombospondin 1 | 469 | 470 | 471 |
| TLR2 | Toll Like Receptor 2 | 472 | 473 | 474 |
| TMEM158 | Transmembrane Protein 158 | 475 | 476 | 477 |
| | (Gene/Pseudogene) | | | |
| TMOD1 | Tropomodulin 1 | 478 | 479 | 480 |
| TNS1 | Tensin 1 | 481 | 482 | 483 |
| TP63 | Tumor Protein P63 | 484 | 485 | 486 |
| TRIM27 | Tripartite Motif Containing 27 | 487 | 488 | 489 |
| TSPAN9 | Tetraspanin 9 | 490 | 491 | 492 |
| TTTY10 | Testis-Specific Transcript, Y- | 493 | 494 | non-coding |
| | Linked 10 | | | |
| TTTY15 | Testis-Specific Transcript, Y- | 496 | 497 | non-coding |
| | Linked 15 | | | |
| U2AF2 | U2 Small Nuclear RNA | 499 | 500 | 501 |
| | Auxiliary Factor 2 | | | |
| UPK3B | Uroplakin 3B | 502 | 503 | 504 |
| USP32P2 | Ubiquitin Specific Peptidase | 505 | 506 | non-coding |
| | 32 Pseudogene 2 | | | |
| USP9Y | Ubiquitin Specific Peptidase 9 | 508 | 509 | 510 |
| | Y-Linked | | | |
| XIST | X Inactive Specific Transcript | 511 | 512 | non-coding |
| XK | X-Linked Kx Blood Group | 514 | 515 | 516 |
| XPO7 | Exportin 7 | 517 | 518 | 519 |
| XRCC2 | X-Ray Repair Cross | 520 | 521 | 522 |
| | Complementing 2 | | | |
| YOD1 | YOD1 Deubiquitinase | 523 | 524 | 525 |
| ZFY | Zinc Finger Protein Y-Linked | 526 | 527 | 528 |
| ZNF143 | Zinc Finger Protein 143 | 529 | 530 | 531 |
| ZNF541 | Zinc Finger Protein 541 | 532 | 533 | 534 |
| ZNF831 | Zinc Finger Protein 831 | 535 | 536 | 537 |

In an aspect, a gene expression classifier comprises all 179 biomarkers in Table 1, Table 2, and Table 3. In an aspect, a gene expression classifier comprises a biomarker selected from the 143 biomarkers in Table 3. In an aspect, a gene expression classifier comprises a biomarker selected from the 36 biomarkers in Table 1 and Table 2; and a biomarker selected from the 143 biomarkers in Table 3. In an aspect, a gene expression classifier comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises ABCC13 and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises ADCK3 and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises GALNT14 and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises GNAI2 and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises PPM1M and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises ZER1 and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers selected from the 179 biomarkers in Table 1, Table 2, and Table 3.

In an aspect, a gene expression classifier comprises ABCC13, ADCK3, GALNT14, GNAI2, PPM1M, and ZER1. In an aspect, a gene expression classifier comprises ABCC13, ADCK3, GALNT14, GNAI2, PPM1M, ZER1, and at least 1, at least 5, at least 10, at least 15, at least 20, or at least 25 biomarkers selected from the 36 biomarkers in Table 1 and Table 2. In an aspect, a gene expression classifier comprises ABCC13, ADCK3, GALNT14, GNAI2, PPM1M, ZER1, and at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, or at least 170 biomarkers selected from the 179 biomarkers in Table 1, Table 2, and Table 3.

In an aspect, the expression level of a biomarker in Table 1, Table 2, and Table 3 is detected using a nucleic acid probe that binds to a nucleic acid sequence having the corresponding full length nucleic acid sequence, or a portion thereof. In an aspect, the expression level of a biomarker in Table 1, Table 2, and Table 3 is detected using a nucleic acid probe that binds to a nucleic acid sequence having at least 80%, at least 85%, at least 90%, at least 93%, at least 95%, at least 98%, at least 99%, at least 99.5%, at least 99.8%, or at least 99.9% sequence identity to the corresponding full length nucleic acid sequence, or a portion thereof.

In an aspect, the expression level of a biomarker in Table 1, Table 2, and Table 3 is detected using an antibody, or a fragment thereof that binds to a protein having an amino acid sequence of the corresponding full length polypeptide sequence. In an aspect, the expression level of a biomarker in Table 1, Table 2, and Table 3 is detected using an antibody, or a fragment thereof that binds to a protein having an amino acid sequence that has at least 80%, at least 85%, at least 90%, at least 93%, at least 95%, at least 98%, at least 99%, at least 99.5%, at least 99.8%, or at least 99.9% sequence identity to the corresponding full length polypeptide sequence.

Comparison of Biomarkers

In one aspect, a control or reference level of a biomarker or a panel of biomarkers is obtained from a single subject. In an aspect, a control or reference level of a biomarker or a panel of biomarkers is obtained from a population of individuals sharing a specific characteristic. In an aspect, a control or reference level of a biomarker or a panel of biomarkers is an assigned value which correlates with the level of a specific control individual or population, although not necessarily measured at the time of assaying the test subject's sample. In an aspect, the control subject or reference of a biomarker or a panel of biomarkers is obtained from a patient (or population) having a non-cancerous nodule.

In one aspect, a control subject or reference is a patient (or population) having a cancerous tumor. In an aspect, a control subject is a subject or population with lung cancer, such as a subject who is a current or former smoker with malignant disease, a subject with a solid lung tumor prior to surgery for removal of same; a subject with a solid lung tumor following surgical removal of the tumor; a subject with a solid lung tumor prior to therapy for same; and a subject with a solid lung tumor during or following therapy for same. In an aspect, controls for purposes of the compositions and methods described herein include any of the following classes of reference human subject with no lung cancer, such as the classes of smoker with non-malignant disease, a former smoker with non-malignant disease (including patients with lung nodules), a non-smoker who has chronic obstructive pulmonary disease (COPD), and a former smoker with COPD. In an aspect, the control subject is a healthy non-smoker with no disease or a healthy smoker with no disease.

In an aspect, differential gene expression can include a comparison of expression between two or more genes or their gene products, or a comparison of the ratios of the expression between two or more genes or their gene products, or even a comparison of two differently processed products of the same gene, which differ between normal subjects, non-health controls and subjects suffering from a disease, specifically cancer, or between various stages of the same disease. In an aspect, differential expression includes both quantitative, as well as qualitative, differences in the temporal or cellular expression pattern in a gene or its expression products among, for example, normal and diseased cells, or among cells which have undergone different disease events or disease stages. In an aspect, differential gene expression is considered to be present when there is a statistically significant difference in gene expression between the subject and control samples.

Methods of Identifying Lung Cancer Risks

The present disclosure provides for, and includes, methods for detecting whether a subject is at risk of lung cancer based on a blood-based multivariate gene expression classifier. The present disclosure further provides for, and includes methods for determining whether to obtain a biopsy in a subject based on a blood-based multivariate gene expression classifier. The present disclosure also provides for, and includes, methods for treating lung cancer based on a blood-based multivariate gene expression classifier.

In an aspect, a method for detecting whether a subject is at risk of lung cancer is provided, the method comprising detecting a lung nodule in the subject, determining the size of the nodule, assigning the subject as being at high risk of lung cancer if the nodule size is larger than 3 cm; assigning the subject as being at low risk of lung cancer if the nodule size is less than 0.8 cm; obtaining or having obtained non-invasively a biological sample from the subject if the nodule size is 0.8 cm to 3.0 cm, detecting the expression levels of biomarkers of a gene expression classifier in the biological sample, comparing the expression levels to a reference, and flagging that the nodule is at high risk or low risk of lung cancer based on the comparing step. In an aspect, the method further comprises obtaining or having obtained a biopsy from the subject if the subject is determined to be at high risk of lung cancer. In an aspect, the method further comprises performing or having performed serial imaging of the subject if the subject is determined to be at low risk of lung cancer. In an aspect, the gene expression classifier is selected per paragraphs [0067]-[0094].

In an aspect, a method for determining whether a subject is at risk of lung cancer is provided, the method comprising obtaining or having obtained non-invasively a biological sample from the subject; detecting expression levels of biomarkers of a gene expression classifier in the biological sample; comparing the expression levels to a reference to determine if the subject is at risk of lung cancer; and providing a recommendation that the subject is at risk of lung cancer based on the comparing step. In an aspect, the gene expression classifier is selected per paragraphs [0067]-[0094].

In an aspect, a method for identifying a subject in need of a biopsy is provided, the method comprising obtaining or having obtained non-invasively a biological sample from the subject; detecting expression levels of biomarkers of a gene expression classifier in the biological sample; comparing the expression levels to a reference to determine if the subject is at risk of lung cancer; and obtaining or having obtained a biopsy from the subject to test for lung cancer if the subject is determined to be at risk of lung cancer based on the comparing step. In an aspect, the gene expression classifier is selected per paragraphs [0067]-[0094]. In an aspect, the method further comprises a step of administering or having administered a lung cancer therapy to the subject if the biopsy shows malignancy. In an aspect, the lung cancer therapy is selected from the group consisting of surgery, chemotherapy, radiation therapy, targeted therapy, and immunotherapy. In one aspect, lung cancer therapy is provided as chemotherapy after surgery. In an aspect, lung cancer therapy is provided as a neoadjuvant treatment, which is providing chemotherapy before radiation or surgery. In one aspect, a surgical treatment for lung cancer is video-assisted thoracoscopic surgery (VATS). In an aspect, chemotherapy is selected from a group consisting of cisplatin (Platinol) or carboplatin (Paraplatin) plus docetaxel (Taxotere), gemcitabine (Gemzar), paclitaxel (Taxol and others), vinorelbine (Navelbine and others), and pemetrexed (Alimta), or a combination thereof. In an aspect, targeted therapy is selected from the group consisting of Erlotinib (Tarceva and others), Afatinib (Gilortif), Gefitinib (Iressa), Bevacizumab (Avastin), Crizotinib (Xalkori), and Ceritinib (Zykadia). In one aspect, immunotherapy is selected from the group consisting of monoclonal antibodies, checkpoint inhibitors, therapeutic vaccines, and adoptive T-cell transfer.

In an aspect, a method for determining whether a subject having one or more lung nodules of the size 0.8 cm to 3.0 cm is at risk of lung cancer is provided, the method comprising obtaining or having obtained non-invasively a biological sample from the subject; detecting expression levels of biomarkers of a gene expression classifier in the biological sample; comparing the expression levels to a reference to determine if the subject is at risk of lung cancer; and flagging whether the subject is at risk of lung cancer based on the comparing step. In an aspect, the gene expression classifier is selected per paragraphs [0067]-[0094]. In an aspect, the expression levels determine whether the one or more lung nodules is malignant or benign. In an aspect, one or more lung nodules of the subject has a size of from 0.5 cm to 3.0 cm, such as from 0.8 cm to 3.0 cm, from 0.5 cm to 2.0 cm, or from 0.8 cm to 2.0 cm.

In an aspect, a method for treating a subject in need of lung cancer therapy is provided, the method comprising obtaining or having obtained non-invasively a biological sample from the subject; detecting expression levels of biomarkers of a gene expression classifier in the biological sample; comparing the expression levels to a reference to determine if the subject is at risk of lung cancer; and administering or having administered a lung cancer therapy if the subject is determined to be at risk of lung cancer based on the comparing step. In an aspect, the gene expression classifier is selected per paragraphs [0067]-[0094]. In an aspect, the lung cancer therapy is selected from the group consisting of surgery, chemotherapy, radiation therapy, targeted therapy, and immunotherapy. In one aspect, lung cancer therapy is provided as chemotherapy after surgery. In an aspect, lung cancer therapy is provided as a neoadjuvant treatment, which is providing chemotherapy before radiation or surgery. In one aspect, a surgical treatment for lung cancer is video-assisted thoracoscopic surgery (VATS). In an aspect, chemotherapy is selected from a group consisting of cisplatin (Platinol) or carboplatin (Paraplatin) plus docetaxel (Taxotere), gemcitabine (Gemzar), paclitaxel (Taxol and others), vinorelbine (Navelbine and others), and pemetrexed (Alimta), or a combination thereof. In an aspect, targeted therapy is selected from the group consisting of Erlotinib (Tarceva and others), Afatinib (Gilortif), Gefitinib (Iressa), Bevacizumab (Avastin), Crizotinib (Xalkori), and Ceritinib (Zykadia). In one aspect, immunotherapy is selected from the group consisting of monoclonal antibodies, checkpoint inhibitors, therapeutic vaccines, and adoptive T-cell transfer.

Kits for Identifying Lung Cancer Risks

The present disclosure provides for, and includes kits for detecting the expression levels of biomarkers for determining whether a subject is at risk of lung cancer. In an aspect, kits of the present disclosure are capable of detecting whether a lung nodule of the size 0.8 cm to 3 cm is malignant or benign.

In an aspect, a kit comprises a probe for detecting the expression levels of biomarkers of a gene expression classifier for determining whether a subject is at risk of lung cancer. In an aspect the gene expression classifier is selected per paragraphs [0067]-[0094].

In one aspect, a kit comprises at least 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, or 175 polynucleotides or oligonucleotides or ligands, wherein each polynucleotide or oligonucleotide or ligand hybridizes to a different gene, gene fragment, gene transcript or expression product in a sample selected from the genes of Table 1, Table 2, and Table 3. In an aspect, an oligonucleotide probe is a synthetic oligonucleotide probe. In one aspect, an oligonucleotide probe is partially complementary to a polynucleotide of interest and forms a duplex structure by hybridization with at least one strand of the polynucleotide of interest. In an aspect, an oligonucleotide probe is fully complementary to a polynucleotide of interest and forms a duplex structure by hybridization with at least one strand of the polynucleotide of interest. In one aspect, an oligonucleotide probe are from 10 to 50 nucleotides in length, such as from 10 to 45 nucleotides in length, from 10 to 40 nucleotides in length, from 10 to 35 nucleotides in length, from 10 to 30 nucleotides in length, from 10 to 25 nucleotides in length, from 10 to 20 nucleotides in length, from 10 to 15 nucleotides in length, from 15 to 50 nucleotides in length, from 15 to 45 nucleotides in length, from 15 to 40 nucleotides in length, from 15 to 35 nucleotides in length, from 15 to 30 nucleotides in length, from 15 to 25 nucleotides in length, from 15 to 20 nucleotides in length, from 20 to 50 nucleotides in length, from 20 to 45 nucleotides in length, from 20 to 40 nucleotides in length, from 20 to 35 nucleotides in length, from 20 to 30 nucleotides in length, from 20 to 25 nucleotides in length, from 25 to 50 nucleotides in length, from 25 to 45 nucleotides in length, from 25 to 40 nucleotides in length, from 25 to 35 nucleotides in length, from 25 to 30 nucleotides in length, from 30 to 50 nucleotides in length, from 30 to 45 nucleotides in length, from 30 to 40 nucleotides in length, from 30 to 35 nucleotides in length, from 35 to 50 nucleotides in length, from 35 to 45 nucleotides in length, from 35 to 40 nucleotides in length, from 40 to 50 nucleotides in length, from 40 to 45 nucleotides in length, or from 45 to 50 nucleotides in length. In an aspect, an oligonucleotide probe has a sequence of a biomarker, complement or fragment thereof, selected from the group of biomarkers listed in Table 1, Table 2, and Table 3. In one aspect, an oligonucleotide probe comprise a fragment of a biomarker selected from the group of biomarkers listed in Table 1, Table 2, and Table 3, or a complement thereof, where the fragment has a length of at least 10 nucleotides, such as at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 90 nucleotides, at least 95 nucleotides, or at least 100 nucleotides. In an aspect, a kit of the present disclosure can be used with the gene expression profiling methods which are known in the art. In one aspect, a kit can be adapted accordingly to suit the method for which they are intended to be used. In one aspect, at least one polynucleotide or oligonucleotide or ligand is attached to a detectable label. In an aspect, each polynucleotide or oligonucleotide is attached to a different detectable label, each capable of being detected independently.

In an aspect, a kit comprises a capture oligonucleotide or ligand, which hybridizes to at least one polynucleotide or oligonucleotide or ligand. In one aspect, a capture oligonucleotide or ligand can include a nucleic acid sequence which is specific for a portion of the oligonucleotide or polynucleotide or ligand which is specific for the gene of interest. In an aspect, a capture ligand can be a peptide or polypeptide which is specific for the ligand to the gene of interest. In one aspect, a capture ligand is an antibody, as in a sandwich ELISA.

In an aspect, a capture oligonucleotide also includes a moiety which allows for binding with a substrate. In one aspect, a substrate can be, without limitation, a plate, bead, slide, well, chip or chamber. In one aspect, a kit comprises a capture oligonucleotide for each different polynucleotide or oligonucleotide which is specific to a gene of interest. In an aspect, each capture oligonucleotide can contain the same moiety which allows for binding with the same substrate.

Detection of biomarkers of the present disclosure can be performed through any methodologies known to those having ordinary skill in the art. Examples of detection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454), enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides.

### EXAMPLES

### Example 1: Sample Collection and Processing

Peripheral venous blood (2.0 ml) is collected in a Qiagen PAXgene Blood RNA Tube by phlebotomy. After sample collection, the PAXgene tubes are immediately inverted 8 to 10 times and placed in a tube rack at room temperature for no more than 3 hours. Samples are then frozen at −20° C., followed by −80° C. where available. No sample is subjected to more than one freeze and thaw cycle. Alternatively, fresh blood not subject to any freeze-thaw cycles are processed within 48 hours of collection.

Total RNA is extracted and purified from each sample using the Qiagen PAXgene Blood miRNA Kit. RNA concentration is measured with the NanoDrop® One Spectrophotomer (Thermo Fisher Scientific), and RNA quality is assessed on the Agilent 2100 Bioanalyzer. Samples are then analyzed using the ThermoFisher Ion GeneStudio S5 Next-Generation Sequencing System. Only samples with RNA yield ≥100 ng, RNA concentration ≥10 ng/μL, RNA integrity numbers of ≥5.0, and no instrument error messages are used for classifier training and validation.

### Example 2: Classifier Training and Analysis

A gene expression classifier consisting of biomarkers listed in Table 1 is trained with measurements from the 565 benign and 136 malignant samples in a training population. See, e.g. Table 4. The genes used in the multivariate classifier are selected using variable selection techniques based on contribution scores from random bootstrap forests and bootstrapped p-values from a generalized regression modeling using a lasso fitting algorithm. The final classifier combines the genes using a logistic model.

Sensitivity, specificity, and negative predictive value are used to evaluate performance of the classifier based on data from the training population. The selected gene expression classifier is then used to make predictions of disease in the validation population, which is an independent data set of samples not used in the training of the classifier. See, e.g. Table 4. Receiver Operating Characteristic (ROC) curve analysis is used to compare the performance of the gene expression classifier with that of the Mayo Clinic model, which is based exclusively on clinical parameters, including age, smoking status, cancer history, and location, size and shape of nodules. See Swensen S J, Silverstein M D, Ilstrup D M, Schleck C D, Edell E S. The probability of malignancy in solitary pulmonary nodules. Application to small radiologically indeterminate nodules. Arch Intern Med. 1997; 157(8):849-855. In addition, the performance of the gene expression classifier is assessed in the low, intermediate and high risk groups as identified by the Mayo Clinic Model. See id.

TABLE 4
Sample demographics for a training population and a validation population

| | Training Population | | | Validation Population | | |
|---|---|---|---|---|---|---|
| Category | Benign | Malignant | All | Benign | Malignant | All |
| Total, n | 565 | 136 | 701 | 169 | 80 | 249 |
| Sex, n | | | | | | |
| Female | 259 | 71 | 330 | 46 | 41 | 87 |
| Male | 306 | 65 | 371 | 123 | 39 | 162 |
| Age, yr | | | | | | |
| Median | 66 | 71 | 67 | 68 | 67 | 67.4 |
| Range | 24-92 | 46-88 | 24-92 | 40-91 | 42-89 | 40-91 |
| Smoking History^{c}, pack years | | | | | | |
| Median | 35 | 45 | 38 | 40 | 42 | 40 |
| Range | 0.2-200 | 2-185 | 0.2-200 | 1-600 | 15-110 | 1-600 |
| Nodule size, cm | | | | | | |
| Median | 1.0 | 1.8 | 1.1 | 0.9 | 1.7 | 1.2 |
| Range | 0.5-3.0 | 0.6-3.0 | 0.5-3.0 | 0.5-2.7 | 0.8-3.0 | 0.5-3.0 |
| Nodule size classification, n | | | | | | |
| 0.5 cm-0.7 cm | 177 | 4 | 181 | 59 | 0 | 59 |
| 0.8 cm-1.0 cm | 144 | 12 | 156 | 42 | 9 | 51 |
| 1.1 cm-2.0 cm | 193 | 71 | 264 | 50 | 45 | 95 |
| 2.1 cm-3.0 cm | 51 | 49 | 100 | 18 | 26 | 44 |
| Histologic diagnosis, n | | | | | | |
| Benign | 565 | 0 | 565 | 169 | 0 | 169 |
| Adenocarcinoma | 0 | 106 | 106 | 0 | 60 | 60 |
| NSCLC | 0 | 0 | 0 | 0 | 1 | 1 |
| Squamous | 0 | 30 | 30 | 0 | 18 | 18 |
| Neoplasm of uncertain | 0 | 0 | 0 | 0 | 1 | 1 |
| behavior | | | | | | |
| Fungal geographic | | | | | | |
| distribution, n | | | | | | |
| Coccidioidomycosis | 24 | 3 | 27 | 0 | 1 | 1 |
| Histoplasmosis/Blastomycosis | 268 | 56 | 324 | 113 | 33 | 146 |
| Histoplasmosis/Blastomycosis/ | 43 | 5 | 48 | 4 | 3 | 7 |
| Coccidioidomycosis | | | | | | |
| N/A | 230 | 72 | 302 | 52 | 43 | 95 |

### Example 3: Comparison of Training Population and Validation Population

Samples are prospectively collected, using the method of Example 1, from current and former smokers with lung nodules found by positive low-dose computed tomography (LDCT) screening at over 60 geographically, ethnically, and point-of-care diverse clinical sites across the United States. Inclusion criteria are ages 21 years old or older, current or past history of smoking tobacco, and having nodule sized between 0.5 cm and 3.0 cm as identified by LDCT or other imaging modalities. Exclusion criteria are as follows: (a) not a candidate for bronchoscopy, biopsy, or surgery; (b) current diagnosis of cancer with treatment.

Nodules are confirmed as either benign or malignant by radiology or by pathologic diagnosis through bronchoscopy, biopsy, and/or lung resection. A representative subset of 701 samples from 57 clinical sites are selected from the sample bank for the population used to train the gene expression classifier in accordance with Example 2. A separate representative subset of 250 samples from 44 clinical sites are selected from the sample bank in a blinded fashion for the population used to validate the performance of the classifier in accordance with Example 2. Among samples from patients with malignant nodules all cancer types are represented, including adenocarcinoma, squamous, NSCLC and other neoplasm, though larger numbers would be needed to confidently distinguish risk of lung cancer for each of these cancer types.

The training and validation populations are selected from the Lung Cancer-Reduce Unnecessary Biopsies Study (LC-RUBS) bank. The LC-RUBS has enrolled over 3000 qualifying subjects from over 60 clinical sites across the United States to explore blood-based RNA biomarkers for lung cancer screening and lung nodule evaluation. The LC-RUBS is designed to evaluate the potential utility of mRNA biomarkers in whole blood for the early detection of lung cancer in patients with lung nodules identified by LDCT or other imaging technologies across heterogeneous sites and geographies within the U.S. screening population. When complete, the LC-RUBS will be the largest prospective, multicenter, nationally representative cohort for the study of biomarkers in patients with 5-30 mm lung nodules to date. The purpose of the manuscript to be submitted is to describe the (1) LC-RUBS cohort and study design, (2) baseline characteristics of enrolled individuals, including demographic and clinical information, fungal geographic distribution, and lung nodule data; and (3) establishment of a biospecimen repository containing peripheral blood samples collected from the LC-RUBS cohort.

The training population is selected with a 40% prevalence based on sample sizes needed for numerical estimation of sensitivity, specificity, and AUC with pre-specified precision. The validation population is selected in a blinded fashion to reflect clinical populations (32% prevalence). Otherwise the two study populations are clinically comparable. There was no significant difference in the distribution or mean values for the Mayo scores, with a mean Mayo score of 0.28 in the training population and 0.31 in the validation population (t-test p-value=0.0662). There are also no significant differences in the individual components of the Mayo Clinic model, with similar values observed between the two populations for age, cancer history, smoking history, nodule size, spiculation, and location. There is a difference in the proportion of samples from males and females, with 53% males in the training population and 65% males in the validation population. There are no significant differences in rates of chronic obstructive pulmonary disease (COPD) or emphysema, with roughly half of samples from patients with COPD (50% training population, 52% validation population) and a third of samples from patients with emphysema (29% training population, 33% validation population).

### Example 4: Gene Expression Classifier Performance

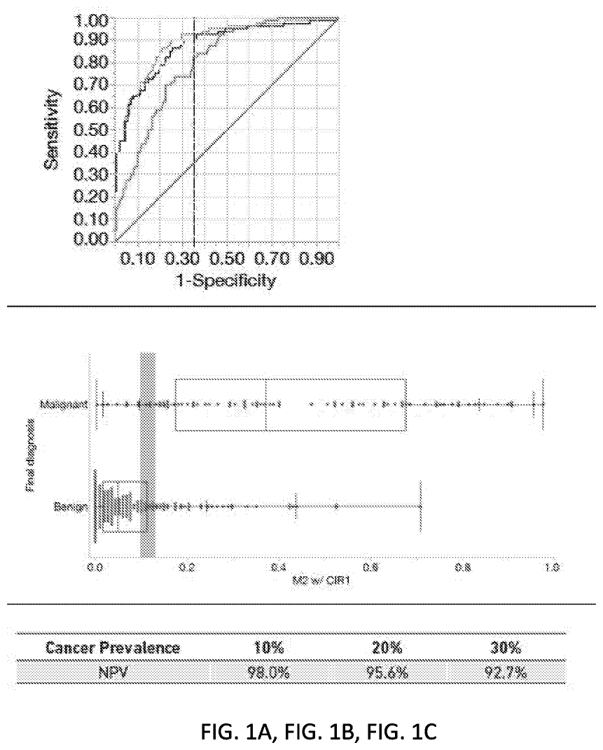

Performance of a gene expression classifier in accordance with Example 2 is compared to the Mayo Clinic model by calculating the area under the ROC curve (AUC), which gives a measure of how well a test performs in identifying true positives and true negatives (FIG. 1A). The gene expression classifier in this example yields an AUC of 0.89 (0.5 cm-3.0 cm nodules), whereas the Mayo Clinic model yielded an AUC of 0.80. The difference in AUC values was significant (p-value=0.0065), indicating that the gene expression classifier of this example, which provides a purely biological assessment of the risk of cancer, significantly improves upon the clinical risk factors assessed in the Mayo Clinic model.

The individual classification of all benign and malignant lung nodules in the validation population is assessed visually using scatter plots (FIG. 1B). Applying the pre-specified threshold to estimate performance results in a sensitivity of 90% (95% CI, 82%-95%) and specificity of 75% (95% CI, 68%-81%) for the gene expression classifier of this example. These results support the ability of the classifier in differentiating benign from malignant lung nodules within the broad size range of 0.5 cm to 3.0 cm.

The probability that nodules identified as benign by the gene expression classifier truly are benign as determined by radiology or by pathologic diagnosis is assessed by calculating the negative predictive value (NPV) for the classifier over a range of cancer prevalence rates (FIG. 1C). This classifier shows NPVs of 98%, 95.6% and 92.7% at cancer prevalence rates of 10%, 20% and 30%, respectively. The high NPVs demonstrated for the classifier over a range of cancer prevalence rates indicate the classifier could be useful in helping rule out unnecessary biopsies for patients with lung nodules identified by LDCT.

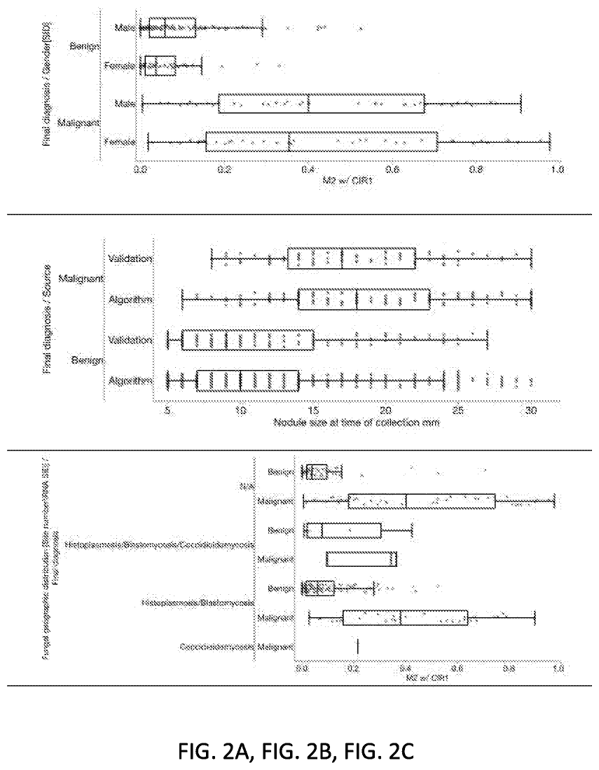

The robustness of the gene expression classifier is examined by evaluating its' performance across a range of clinical variables. As revealed by the scatter plots shown in FIG. 2A, FIG. 2B, and FIG. 2C, no difference in classifier performance is observed based on sex, nodule size, or fungal geographic distribution. These results indicate that clinical variables, which reflect the heterogeneity of patients, do not influence the performance of the gene expression classifier. Despite potential biological differences between patients in the validation population, this classifier is able to reliably distinguish between benign and malignant lung nodules.

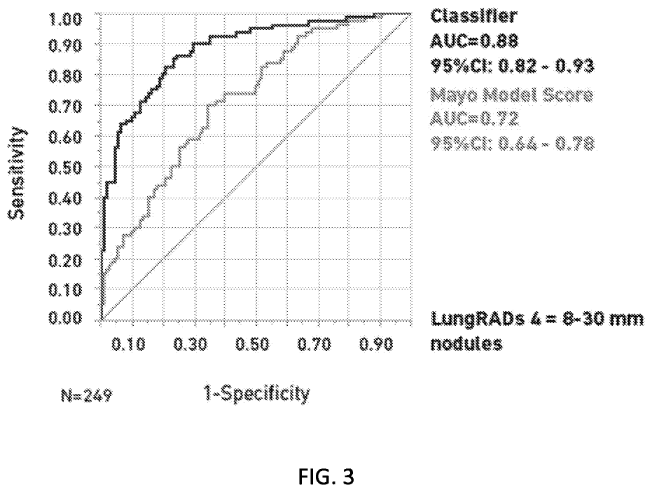

The more clinically challenging intermediate nodule size cases within the validation population are evaluated by calculating AUC values for the subset of samples from subjects with lung nodules between 8 cm and 30 cm and comparing those to the AUC values calculated for the entire validation population of subjects with lung nodules between 5 cm and 30 cm (FIG. 3). The gene expression classifier yields an AUC of 0.88 for the subset of samples from subjects with lung nodules between 8 cm and 30 cm, which is comparable to the AUC of 0.89 calculated for the entire validation population. By comparison, the Mayo Clinic model yields an AUC of 0.72 for the subset of samples from subjects with lung nodules between 8 cm and 30 cm, which is significantly lower than the AUC of 0.80 calculated for the entire validation population. These results show that this gene expression classifier performs equally well, while the performance of the Mayo Clinic model significantly declined for the more clinically challenging cases within the validation population.

### Example 5: A Second Expression Classifier

A second gene expression classifier consisting of a panel of biomarkers including ABCC13, AMD1, ANKHD1, CIR1, CLCN4, CLK3, CTSH, DNAJB1, EPHX2, GALNT14, GNAI2, GSR, IFI27, INO80C, LGR6, PDCD6IP, PNPLA6, PSMB4, RBPMS2, SCAF4, SLC25A20, SNRPD3, STAMBP, TMEM8A, UBAP2, VPS29, ZER1 is trained with measurements from benign and malignant samples in a training population. The selected gene expression classifier is then used to make predictions of disease in the validation population, which is an independent data set of samples not used in the training of the classifier.

### Example 6: A Third Expression Classifier

A second gene expression classifier consisting of a panel of biomarkers including AMD1, CIR1, CLCN4, CLK3, GNAI2, GSR, HDAC3, ITPR1, LGR6, LILRA5, PPM1M, PUM1, PSMB4, SCAF4, SLC25A20, SNRPD3, TPR, UBAP2, WDFY3, ZER1 is trained with measurements from benign and malignant samples in a training population. The selected gene expression classifier is then used to make predictions of disease in the validation population, which is an independent data set of samples not used in the training of the classifier.

From the foregoing, it will be appreciated that the present invention can be embodied in various ways, which include but not limited to the following:

Embodiment 1. A method comprising: determining whether a subject is at risk of lung cancer by: obtaining or having obtained non-invasively a biological sample from the subject; detecting expression level of ABCC13 in the biological sample; and comparing the expression level of ABCC13 to a reference level to determine if the subject is at risk of lung cancer; providing a recommendation that the subject is at risk of lung cancer based on said comparing.

Embodiment 2. A method comprising: determining whether a subject is at risk of lung cancer by: obtaining or having obtained non-invasively a biological sample from the subject; detecting expression level of ABCC13 in the biological sample; and comparing the expression level of ABCC13 to a reference level to determine if the subject is at risk of lung cancer; if the subject is at risk of lung cancer, obtaining or having obtained a biopsy from the subject to test for lung cancer.

Embodiment 3. The method of embodiment 1 or 2, further comprising detecting expression level of a second biomarker in the sample to establish a biomarker expression profile for the subject, the second biomarker is selected from the group consisting of: ADCK3, GALNT14, GNAI2, PPM1M, and ZER1, and where said comparing further comprises comparing the biomarker expression profile to a reference expression profile to determine if the subject is at risk of lung cancer.

Embodiment 4. The method of embodiment 1 or 2, further comprising detecting expression level of additional biomarkers in the sample to establish a biomarker expression profile for the subject, the additional biomarkers comprise two biomarkers selected from the group consisting of: ADCK3, GALNT14, GNAI2, PPM1M, and ZER1, and where said comparing further comprises comparing the biomarker expression profile to a reference expression profile to determine if the subject is at risk of lung cancer.

Embodiment 5. The method of embodiment 1 or 2, further comprising detecting expression level of additional biomarkers in the sample to establish a biomarker expression profile for the subject, the additional biomarkers comprise three biomarkers selected from the group consisting of: ADCK3, GALNT14, GNAI2, PPM1M, and ZER1, and where said comparing further comprises comparing the biomarker expression profile to a reference expression profile to determine if the subject is at risk of lung cancer.

Embodiment 6. The method of embodiment 1 or 2, further comprising detecting expression level of additional biomarkers in the sample to establish a biomarker expression profile for the subject, the additional biomarkers comprise four biomarkers selected from the group consisting of: ADCK3, GALNT14, GNAI2, PPM1M, and ZER1, and where said comparing further comprises comparing the biomarker expression profile to a reference expression profile to determine if the subject is at risk of lung cancer.

Embodiment 7. The method of embodiment 1 or 2, further comprising detecting expression level of additional biomarkers in the sample to establish a biomarker expression profile for the subject, the additional biomarkers comprise ADCK3, GALNT14, GNAI2, PPM1M, and ZER1, and where said comparing further comprises comparing the biomarker expression profile to a reference expression profile to determine if the subject is at risk of lung cancer.

Embodiment 8. The method of embodiment 1 or 2, where the expression level of ABCC13 is detected using a nucleic acid probe that binds to the nucleic acid sequence of SEQ ID NO: 2.

Embodiment 9. The method of embodiment 1 or 2, where the expression level of ABCC13 is detected using a nucleic acid probe comprising nucleic acid sequence of SEQ ID NO: 1, complements thereof, or fragments thereof having at least 15 nucleotides.

Embodiment 10. The method of embodiment 1 or 2, where the expression level of ABCC13 is detected using an antibody, or a fragment thereof, that binds to a protein having an amino acid sequence of SEQ ID NO: 3.

Embodiment 11. A method comprising: determining whether a subject is at risk of lung cancer by: obtaining or having obtained non-invasively a biological sample from the subject; detecting expression level of ADCK3 in the biological sample; and comparing the expression level of ADCK3 to a reference level to determine if the subject is at risk of lung cancer; providing a recommendation that the subject is at risk of lung cancer based on said comparing.

Embodiment 12. A method comprising: determining whether a subject is at risk of lung cancer by: obtaining or having obtained non-invasively a biological sample from the subject; detecting expression level of ADCK3 in the biological sample; and comparing the expression level of ADCK3 to a reference level to determine if the subject is at risk of lung cancer; if the subject is at risk of lung cancer, obtaining or having obtained a biopsy from the subject to test for lung cancer.

Embodiment 13. The method of embodiment 11 or 12, further comprising detecting expression level of a second biomarker in the sample to establish a biomarker expression profile for the subject, the second biomarker is selected from the group consisting of: ABCC13, GALNT14, GNAI2, PPM1M, and ZER1, and where said comparing further comprises comparing the biomarker expression profile to a reference expression profile to determine if the subject is at risk of lung cancer.

Embodiment 14. The method of embodiment 11 or 12, further comprising detecting expression level of additional biomarkers in the sample to establish a biomarker expression profile for the subject, the additional biomarkers comprise two biomarkers selected from the group consisting of: ABCC13, GALNT14, GNAI2, PPM1M, and ZER1, and where said comparing further comprises comparing the biomarker expression profile to a reference expression profile to determine if the subject is at risk of lung cancer.

Embodiment 15. The method of embodiment 11 or 12, further comprising detecting expression level of additional biomarkers in the sample to establish a biomarker expression profile for the subject, the additional biomarkers comprise three biomarkers selected from the group consisting of: ABCC13, GALNT14, GNAI2, PPM1M, and ZER1, and where said comparing further comprises comparing the biomarker expression profile to a reference expression profile to determine if the subject is at risk of lung cancer.

Embodiment 16. The method of embodiment 11 or 12, further comprising detecting expression level of additional biomarkers in the sample to establish a biomarker expression profile for the subject, the additional biomarkers comprise four biomarkers selected from the group consisting of: ABCC13, GALNT14, GNAI2, PPM1M, and ZER1, and where said comparing further comprises comparing the biomarker expression profile to a reference expression profile to determine if the subject is at risk of lung cancer.

Embodiment 17. The method of embodiment 11 or 12, further comprising detecting expression level of additional biomarkers in the sample to establish a biomarker expression profile for the subject, the additional biomarkers comprise ABCC13, GALNT14, GNAI2, PPM1M, and ZER1, and where said comparing further comprises comparing the biomarker expression profile to a reference expression profile to determine if the subject is at risk of lung cancer.

Embodiment 18. The method of embodiment 11 or 12, where the expression level of ADCK3 is detected using a nucleic acid probe that binds to the nucleic acid sequence of SEQ ID NO: 5.

Embodiment 19. The method of embodiment 11 or 12, where the expression level of ADCK3 is detected using a nucleic acid probe comprising nucleic acid sequence of SEQ ID NO: 4, complements thereof, or fragments thereof having at least 15 nucleotides.

Embodiment 20. The method of embodiment 11 or 12, where the expression level of ADCK3 is detected using an antibody, or a fragment thereof, that binds to a protein having an amino acid sequence of SEQ ID NO: 6.

Embodiment 21. A method comprising: determining whether a subject is at risk of lung cancer by: obtaining or having obtained non-invasively a biological sample from the subject; detecting expression level of GALNT14 in the biological sample; and comparing the expression level of GALNT14 to a reference level to determine if the subject is at risk of lung cancer; providing a recommendation that the subject is at risk of lung cancer based on said comparing.

Embodiment 22. A method comprising: determining whether a subject is at risk of lung cancer by: obtaining or having obtained non-invasively a biological sample from the subject; detecting expression level of GALNT14 in the biological sample; and comparing the expression level of GALNT14 to a reference level to determine if the subject is at risk of lung cancer; if the subject is at risk of lung cancer, obtaining or having obtained a biopsy from the subject to test for lung cancer.

Embodiment 23. The method of embodiment 21 or 22, further comprising detecting expression level of a second biomarker in the sample to establish a biomarker expression profile for the subject, the second biomarker is selected from the group consisting of: ABCC13, ADCK3, GNAI2, PPM1M, and ZER1, and where said comparing further comprises comparing the biomarker expression profile to a reference expression profile to determine if the subject is at risk of lung cancer.

Embodiment 24. The method of embodiment 21 or 22, further comprising detecting expression level of additional biomarkers in the sample to establish a biomarker expression profile for the subject, the additional biomarkers comprise two biomarkers selected from the group consisting of: ABCC13, ADCK3, GNAI2, PPM1M, and ZER1, and where said comparing further comprises comparing the biomarker expression profile to a reference expression profile to determine if the subject is at risk of lung cancer.

Embodiment 25. The method of embodiment 21 or 22, further comprising detecting expression level of additional biomarkers in the sample to establish a biomarker expression profile for the subject, the additional biomarkers comprise three biomarkers selected from the group consisting of: ABCC13, ADCK3, GNAI2, PPM1M, and ZER1, and where said comparing further comprises comparing the biomarker expression profile to a reference expression profile to determine if the subject is at risk of lung cancer.

Embodiment 26. The method of embodiment 21 or 22, further comprising detecting expression level of additional biomarkers in the sample to establish a biomarker expression profile for the subject, the additional biomarkers comprise four biomarkers selected from the group consisting of: ABCC13, ADCK3, GNAI2, PPM1M, and ZER1, and where said comparing further comprises comparing the biomarker expression profile to a reference expression profile to determine if the subject is at risk of lung cancer.

Embodiment 27. The method of embodiment 21 or 22, further comprising detecting expression level of additional biomarkers in the sample to establish a biomarker expression profile for the subject, the additional biomarkers comprise ABCC13, ADCK3, GNAI2, PPM1M, and ZER1, and where said comparing further comprises comparing the biomarker expression profile to a reference expression profile to determine if the subject is at risk of lung cancer.

Embodiment 28. The method of embodiment 21 or 22, where the expression level of GALNT14 is detected using a nucleic acid probe that binds to the nucleic acid sequence of SEQ ID NO: 23.

Embodiment 29. The method of embodiment 21 or 22, where the expression level of GALNT14 is detected using a nucleic acid probe comprising nucleic acid sequence of SEQ ID NO: 22, complements thereof, or fragments thereof having at least 15 nucleotides.

Embodiment 30. The method of embodiment 21 or 22, where the expression level of GALNT14 is detected using an antibody, or a fragment thereof, that binds to a protein having an amino acid sequence of SEQ ID NO: 24.

Embodiment 31. A method comprising: determining whether a subject is at risk of lung cancer by: obtaining or having obtained non-invasively a biological sample from the subject; detecting expression level of GNAI2 in the biological sample; and comparing the expression level of GNAI2 to a reference level to determine if the subject is at risk of lung cancer; providing a recommendation that the subject is at risk of lung cancer based on said comparing.

Embodiment 32. A method comprising: determining whether a subject is at risk of lung cancer by: obtaining or having obtained non-invasively a biological sample from the subject; detecting expression level of GNAI2 in the biological sample; and comparing the expression level of GNAI2 to a reference level to determine if the subject is at risk of lung cancer; if the subject is at risk of lung cancer, obtaining or having obtained a biopsy from the subject to test for lung cancer.

Embodiment 33. The method of embodiment 31 or 32, further comprising detecting expression level of a second biomarker in the sample to establish a biomarker expression profile for the subject, the second biomarker is selected from the group consisting of: ABCC13, ADCK3, GALNT14, PPM1M, and ZER1, and where said comparing further comprises comparing the biomarker expression profile to a reference expression profile to determine if the subject is at risk of lung cancer.

Embodiment 34. The method of embodiment 31 or 32, further comprising detecting expression level of additional biomarkers in the sample to establish a biomarker expression profile for the subject, the additional biomarkers comprise two biomarkers selected from the group consisting of: ABCC13, ADCK3, GALNT14, PPM1M, and ZER1, and where said comparing further comprises comparing the biomarker expression profile to a reference expression profile to determine if the subject is at risk of lung cancer.

Embodiment 35. The method of embodiment 31 or 32, further comprising detecting expression level of additional biomarkers in the sample to establish a biomarker expression profile for the subject, the additional biomarkers comprise three biomarkers selected from the group consisting of: ABCC13, ADCK3, GALNT14, PPM1M, and ZER1, and where said comparing further comprises comparing the biomarker expression profile to a reference expression profile to determine if the subject is at risk of lung cancer.

Embodiment 36. The method of embodiment 31 or 32, further comprising detecting expression level of additional biomarkers in the sample to establish a biomarker expression profile for the subject, the additional biomarkers comprise four biomarkers selected from the group consisting of: ABCC13, ADCK3, GALNT14, PPM1M, and ZER1, and where said comparing further comprises comparing the biomarker expression profile to a reference expression profile to determine if the subject is at risk of lung cancer.

Embodiment 37. The method of embodiment 31 or 32, further comprising detecting expression level of additional biomarkers in the sample to establish a biomarker expression profile for the subject, the additional biomarkers comprise ABCC13, ADCK3, GALNT14, PPM1M, and ZER1, and where said comparing further comprises comparing the biomarker expression profile to a reference expression profile to determine if the subject is at risk of lung cancer.

Embodiment 38. The method of embodiment 31 or 32, where the expression level of GNAI2 is detected using a nucleic acid probe that binds to the nucleic acid sequence of SEQ ID NO: 26.

Embodiment 39. The method of embodiment 31 or 32, where the expression level of GNAI2 is detected using a nucleic acid probe comprising nucleic acid sequence of SEQ ID NO: 25, complements thereof, or fragments thereof having at least 15 nucleotides.

Embodiment 40. The method of embodiment 31 or 32, where the expression level of GNAI2 is detected using an antibody, or a fragment thereof, that binds to a protein having an amino acid sequence of SEQ ID NO: 27.

Embodiment 41. A method comprising: determining whether a subject is at risk of lung cancer by: obtaining or having obtained non-invasively a biological sample from the subject; detecting expression level of PPM1M in the biological sample; and comparing the expression level of PPM1M to a reference level to determine if the subject is at risk of lung cancer; providing a recommendation that the subject is at risk of lung cancer based on said comparing.

Embodiment 42. A method comprising: determining whether a subject is at risk of lung cancer by: obtaining or having obtained non-invasively a biological sample from the subject; detecting expression level of PPM1M in the biological sample; and comparing the expression level of PPM1M to a reference level to determine if the subject is at risk of lung cancer; if the subject is at risk of lung cancer, obtaining or having obtained a biopsy from the subject to test for lung cancer.

Embodiment 43. The method of embodiment 41 or 42, further comprising detecting expression level of a second biomarker in the sample to establish a biomarker expression profile for the subject, the second biomarker is selected from the group consisting of: ABCC13, ADCK3, GALNT14, GNAI2, and ZER1, and where said comparing further comprises comparing the biomarker expression profile to a reference expression profile to determine if the subject is at risk of lung cancer.

Embodiment 44. The method of embodiment 41 or 42, further comprising detecting expression level of additional biomarkers in the sample to establish a biomarker expression profile for the subject, the additional biomarkers comprise two biomarkers selected from the group consisting of: ABCC13, ADCK3, GALNT14, GNAI2, and ZER1, and where said comparing further comprises comparing the biomarker expression profile to a reference expression profile to determine if the subject is at risk of lung cancer.

Embodiment 45. The method of embodiment 41 or 42, further comprising detecting expression level of additional biomarkers in the sample to establish a biomarker expression profile for the subject, the additional biomarkers comprise three biomarkers selected from the group consisting of: ABCC13, ADCK3, GALNT14, GNAI2, and ZER1, and where said comparing further comprises comparing the biomarker expression profile to a reference expression profile to determine if the subject is at risk of lung cancer.

Embodiment 46. The method of embodiment 41 or 42, further comprising detecting expression level of additional biomarkers in the sample to establish a biomarker expression profile for the subject, the additional biomarkers comprise four biomarkers selected from the group consisting of: ABCC13, ADCK3, GALNT14, GNAI2, and ZER1, and where said comparing further comprises comparing the biomarker expression profile to a reference expression profile to determine if the subject is at risk of lung cancer.

Embodiment 47. The method of embodiment 41 or 42, further comprising detecting expression level of additional biomarkers in the sample to establish a biomarker expression profile for the subject, the additional biomarkers comprise ABCC13, ADCK3, GALNT14, GNAI2, and ZER1, and where said comparing further comprises comparing the biomarker expression profile to a reference expression profile to determine if the subject is at risk of lung cancer.

Embodiment 48. The method of embodiment 41 or 42, where the expression level of PPM1M is detected using a nucleic acid probe that binds to the nucleic acid sequence of SEQ ID NO: 92.

Embodiment 49. The method of embodiment 41 or 42, where the expression level of PPM1M is detected using a nucleic acid probe comprising nucleic acid sequence of SEQ ID NO: 91, complements thereof, or fragments thereof having at least 15 nucleotides.

Embodiment 50. The method of embodiment 41 or 42, where the expression level of PPM1M is detected using an antibody, or a fragment thereof, that binds to a protein having an amino acid sequence of SEQ ID NO: 93.

Embodiment 51. A method comprising: determining whether a subject is at risk of lung cancer by: obtaining or having obtained non-invasively a biological sample from the subject; detecting expression level of ZER1 in the biological sample; and comparing the expression level of ZER1 to a reference level to determine if the subject is at risk of lung cancer; providing a recommendation that the subject is at risk of lung cancer based on said comparing.

Embodiment 52. A method comprising: determining whether a subject is at risk of lung cancer by: obtaining or having obtained non-invasively a biological sample from the subject; detecting expression level of ZER1 in the biological sample; and comparing the expression level of ZER1 to a reference level to determine if the subject is at risk of lung cancer; if the subject is at risk of lung cancer, obtaining or having obtained a biopsy from the subject to test for lung cancer.

Embodiment 53. The method of embodiment 51 or 52, further comprising detecting expression level of a second biomarker in the sample to establish a biomarker expression profile for the subject, the second biomarker is selected from the group consisting of: ABCC13, ADCK3, GALNT14, GNAI2, and PPM1M, and where said comparing further comprises comparing the biomarker expression profile to a reference expression profile to determine if the subject is at risk of lung cancer.

Embodiment 54. The method of embodiment 51 or 52, further comprising detecting expression level of additional biomarkers in the sample to establish a biomarker expression profile for the subject, the additional biomarkers comprise two biomarkers selected from the group consisting of: ABCC13, ADCK3, GALNT14, GNAI2, and PPM1M, and where said comparing further comprises comparing the biomarker expression profile to a reference expression profile to determine if the subject is at risk of lung cancer.

Embodiment 55. The method of embodiment 51 or 52, further comprising detecting expression level of additional biomarkers in the sample to establish a biomarker expression profile for the subject, the additional biomarkers comprise three biomarkers selected from the group consisting of: ABCC13, ADCK3, GALNT14, GNAI2, and PPM1M, and where said comparing further comprises comparing the biomarker expression profile to a reference expression profile to determine if the subject is at risk of lung cancer.

Embodiment 56. The method of embodiment 51 or 52, further comprising detecting expression level of additional biomarkers in the sample to establish a biomarker expression profile for the subject, the additional biomarkers comprise four biomarkers selected from the group consisting of: ABCC13, ADCK3, GALNT14, GNAI2, and PPM1M, and where said comparing further comprises comparing the biomarker expression profile to a reference expression profile to determine if the subject is at risk of lung cancer.

Embodiment 57. The method of embodiment 51 or 52, further comprising detecting expression level of additional biomarkers in the sample to establish a biomarker expression profile for the subject, the additional biomarkers comprise ABCC13, ADCK3, GALNT14, GNAI2, and PPM1M, and where said comparing further comprises comparing the biomarker expression profile to a reference expression profile to determine if the subject is at risk of lung cancer.

Embodiment 58. The method of embodiment 51 or 52, where the expression level of ZER1 is detected using a nucleic acid probe that binds to the nucleic acid sequence of SEQ ID NO: 68.

Embodiment 59. The method of embodiment 51 or 52, where the expression level of ZER1 is detected using a nucleic acid probe comprising nucleic acid sequence of SEQ ID NO: 67, complements thereof, or fragments thereof having at least 15 nucleotides.

Embodiment 60. The method of embodiment 51 or 52, where the expression level of ZER1 is detected using an antibody, or a fragment thereof, that binds to a protein having an amino acid sequence of SEQ ID NO: 69.

Embodiment 61. The method of any one of embodiments 1 to 60, further comprising detecting expression level of one or more biomarkers selected from the group consisting of AMD1, CLCN4, PDCD6IP, PUM1, RBPMS2, and UBAP2 to establish a biomarker expression profile for the subject.

Embodiment 62. The method of any one of embodiments 1 to 60, further comprising detecting expression level of two or more biomarkers selected from the group consisting of AMD1, CLCN4, PDCD6IP, PUM1, RBPMS2, and UBAP2 to establish a biomarker expression profile for the subject.

Embodiment 63. The method of any one of embodiments 1 to 60, further comprising detecting expression level of three or more biomarkers selected from the group consisting of AMD1, CLCN4, PDCD6IP, PUM1, RBPMS2, and UBAP2 to establish a biomarker expression profile for the subject.

Embodiment 64. The method of any one of embodiments 1 to 60, further comprising detecting expression level of four or more biomarkers selected from the group consisting of AMD1, CLCN4, PDCD6IP, PUM1, RBPMS2, and UBAP2 to establish a biomarker expression profile for the subject.

Embodiment 65. The method of any one of embodiments 1 to 60, further comprising detecting expression level of five or more biomarkers selected from the group consisting of AMD1, CLCN4, PDCD6IP, PUM1, RBPMS2, and UBAP2 to establish a biomarker expression profile for the subject.

Embodiment 66. The method of any one of embodiments 1 to 60, further comprising detecting expression level of AMD1, CLCN4, PDCD6IP, PUM1, RBPMS2, and UBAP2 to establish a biomarker expression profile for the subject.

Embodiment 67. The method of any one of embodiments 61 to 66, where the expression level of AMD1 is detected using a nucleic acid probe comprising nucleic acid sequence of SEQ ID NO: 7, complements thereof, or fragments thereof having at least 15 nucleotides.

Embodiment 68. The method of any one of embodiments 61 to 66, where the expression level of CLCN4 is detected using a nucleic acid probe comprising nucleic acid sequence of SEQ ID NO: 73, complements thereof, or fragments thereof having at least 15 nucleotides.

Embodiment 69. The method of any one of embodiments 61 to 66, where the expression level of PDCD6IP is detected using a nucleic acid probe comprising nucleic acid sequence of SEQ ID NO: 40, complements thereof, or fragments thereof having at least 15 nucleotides.

Embodiment 70. The method of any one of embodiments 61 to 66, where the expression level of PUM1 is detected using a nucleic acid probe comprising nucleic acid sequence of SEQ ID NO: 94, complements thereof, or fragments thereof having at least 15 nucleotides.

Embodiment 71. The method of any one of embodiments 61 to 66, where the expression level of RBPMS2 is detected using a nucleic acid probe comprising nucleic acid sequence of SEQ ID NO: 97, complements thereof, or fragments thereof having at least 15 nucleotides.

Embodiment 72. The method of any one of embodiments 61 to 66, where the expression level of UBAP2 is detected using a nucleic acid probe comprising nucleic acid sequence of SEQ ID NO: 58, complements thereof, or fragments thereof having at least 15 nucleotides.

Embodiment 73. The method of any one of embodiments 1 to 72, further comprising detecting expression level of a set of biomarkers comprising one or more biomarkers selected from the group consisting of ANKHD1, CIR1, CLK3, CTSH, DNAJB1, EPHX2, GSR, HDAC3, IFI27, INO80C, ITPR1, LGR6, LILRA5, PNPLA6, PSMB4, SCAF4, SLC25A20, SNRPD3, STAMBP, TMEM8A, TPR, VNN1, VPS29, and WDFY3 to establish a biomarker expression profile for the subject.

Embodiment 74. The method of embodiment 73, where the set of biomarkers comprises five or more biomarkers.

Embodiment 75. The method of embodiment 73, where the set of biomarkers comprises ten or more biomarkers.

Embodiment 76. The method of embodiment 73, where the set of biomarkers comprises fifteen or more biomarkers.

Embodiment 77. The method of embodiment 73, where the set of biomarkers comprises twenty or more biomarkers.

Embodiment 78. The method of embodiment 73, where the set of biomarkers comprises all the biomarkers.

Embodiment 79. The method of any one of embodiments 73 to 78, where the expression level of ANKHD1 is detected using a nucleic acid probe comprising nucleic acid sequence of SEQ ID NO: 70, complements thereof, or fragments thereof having at least 15 nucleotides.

Embodiment 80. The method of any one of embodiments 73 to 78, where the expression level of CIR1 is detected using a nucleic acid probe comprising nucleic acid sequence of SEQ ID NO: 10, complements thereof, or fragments thereof having at least 15 nucleotides.

Embodiment 81. The method of any one of embodiments 73 to 78, where the expression level of CLK3 is detected using a nucleic acid probe comprising nucleic acid sequence of SEQ ID NO: 13, complements thereof, or fragments thereof having at least 15 nucleotides.

Embodiment 82. The method of any one of embodiments 73 to 78, where the expression level of CTSH is detected using a nucleic acid probe comprising nucleic acid sequence of SEQ ID NO: 76, complements thereof, or fragments thereof having at least 15 nucleotides.

Embodiment 83. The method of any one of embodiments 73 to 78, where the expression level of DNAJB1 is detected using a nucleic acid probe comprising nucleic acid sequence of SEQ ID NO: 16, complements thereof, or fragments thereof having at least 15 nucleotides.

Embodiment 84. The method of any one of embodiments 73 to 78, where the expression level of EPHX2 is detected using a nucleic acid probe comprising nucleic acid sequence of SEQ ID NO: 19, complements thereof, or fragments thereof having at least 15 nucleotides.

Embodiment 85. The method of any one of embodiments 73 to 78, where the expression level of GSR is detected using a nucleic acid probe comprising nucleic acid sequence of SEQ ID NO: 28, complements thereof, or fragments thereof having at least 15 nucleotides.

Embodiment 86. The method of any one of embodiments 73 to 78, where the expression level of HDAC3 is detected using a nucleic acid probe comprising nucleic acid sequence of SEQ ID NO: 79, complements thereof, or fragments thereof having at least 15 nucleotides.

Embodiment 87. The method of any one of embodiments 73 to 78, where the expression level of IFI27 is detected using a nucleic acid probe comprising nucleic acid sequence of SEQ ID NO: 31, complements thereof, or fragments thereof having at least 15 nucleotides.

Embodiment 88. The method of any one of embodiments 73 to 78, where the expression level of INO80C is detected using a nucleic acid probe comprising nucleic acid sequence of SEQ ID NO: 34, complements thereof, or fragments thereof having at least 15 nucleotides.

Embodiment 89. The method of any one of embodiments 73 to 78, where the expression level of ITPR1 is detected using a nucleic acid probe comprising nucleic acid sequence of SEQ ID NO: 82, complements thereof, or fragments thereof having at least 15 nucleotides.

Embodiment 90. The method of any one of embodiments 73 to 78, where the expression level of LGR6 is detected using a nucleic acid probe comprising nucleic acid sequence of SEQ ID NO: 37, complements thereof, or fragments thereof having at least 15 nucleotides.

Embodiment 91. The method of any one of embodiments 73 to 78, where the expression level of LILRA5 is detected using a nucleic acid probe comprising nucleic acid sequence of SEQ ID NO: 85, complements thereof, or fragments thereof having at least 15 nucleotides.

Embodiment 92. The method of any one of embodiments 73 to 78, where the expression level of PNPLA6 is detected using a nucleic acid probe comprising nucleic acid sequence of SEQ ID NO: 88, complements thereof, or fragments thereof having at least 15 nucleotides.

Embodiment 93. The method of any one of embodiments 73 to 78, where the expression level of PSMB4 is detected using a nucleic acid probe comprising nucleic acid sequence of SEQ ID NO: 43, complements thereof, or fragments thereof having at least 15 nucleotides.

Embodiment 94. The method of any one of embodiments 73 to 78, where the expression level of SCAF4 is detected using a nucleic acid probe comprising nucleic acid sequence of SEQ ID NO: 46, complements thereof, or fragments thereof having at least 15 nucleotides.

Embodiment 95. The method of any one of embodiments 73 to 78, where the expression level of SLC25A20 is detected using a nucleic acid probe comprising nucleic acid sequence of SEQ ID NO: 100, complements thereof, or fragments thereof having at least 15 nucleotides.

Embodiment 96. The method of any one of embodiments 73 to 78, where the expression level of SNRPD3 is detected using a nucleic acid probe comprising nucleic acid sequence of SEQ ID NO: 49, complements thereof, or fragments thereof having at least 15 nucleotides.

Embodiment 97. The method of any one of embodiments 73 to 78, where the expression level of STAMBP is detected using a nucleic acid probe comprising nucleic acid sequence of SEQ ID NO: 52, complements thereof, or fragments thereof having at least 15 nucleotides.

Embodiment 98. The method of any one of embodiments 73 to 78, where the expression level of TMEM8A is detected using a nucleic acid probe comprising nucleic acid sequence of SEQ ID NO: 55, complements thereof, or fragments thereof having at least 15 nucleotides.

Embodiment 99. The method of any one of embodiments 73 to 78, where the expression level of TPR is detected using a nucleic acid probe comprising nucleic acid sequence of SEQ ID NO: 103, complements thereof, or fragments thereof having at least 15 nucleotides.

Embodiment 100. The method of any one of embodiments 73 to 78, where the expression level of VNN1 is detected using a nucleic acid probe comprising nucleic acid sequence of SEQ ID NO: 61, complements thereof, or fragments thereof having at least 15 nucleotides.

Embodiment 101. The method of any one of embodiments 73 to 78, where the expression level of VPS29 is detected using a nucleic acid probe comprising nucleic acid sequence of SEQ ID NO: 64, complements thereof, or fragments thereof having at least 15 nucleotides.

Embodiment 102. The method of any one of embodiments 73 to 78, where the expression level of WDFY3 is detected using a nucleic acid probe comprising nucleic acid sequence of SEQ ID NO: 106, complements thereof, or fragments thereof having at least 15 nucleotides.

Embodiment 103. The method of any one of embodiments 1 to 102, where the biological sample is blood.

Embodiment 104. The method of any one of embodiments 1 to 103, where the subject is a human.

Embodiment 105. The method of any one of embodiments 1 to 104, where the subject has one or more lung nodules suspect of being cancerous.

Embodiment 106. The method of embodiment 105, where the one or more lung nodules have diameters in the range of 0.8 cm to 2 cm.

Embodiment 107. The method of any one of embodiments 1 to 106, where the lung cancer is non-small cell lung cancer (NSCLC).

Embodiment 108. The method of embodiment 107, where the NSCLC is a stage I NSCLC.

Embodiment 109. The method of embodiment 107, where the NSCLC is a stage II NSCLC.

Embodiment 110. The method of embodiment 107, where the NSCLC is a mixture of early and late stages and types of NSCLC.

Embodiment 111. The method of any one of embodiments 1 to 106, where the lung cancer is lung adenocarcinoma.

Embodiment 112. The method of any one of embodiments 1 to 106, where the lung cancer is lung squamous cell carcinoma.

Embodiment 113. The method of any one of embodiments 1 to 106, where the lung cancer is a lung carcinoid tumor.

Embodiment 114. The method of embodiment 113, where the lung carcinoid tumor is a typical carcinoid tumor of the lung.

Embodiment 115. The method of embodiment 113, where the lung carcinoid tumor is an atypical carcinoid tumor of the lung.

Embodiment 116. The method of any one of embodiments 1 to 106, where the lung cancer is adenosquamous carcinoma of the lung.

Embodiment 117. The method of any one of embodiments 2, 12, 22, 32, 42, and 52, further comprising the step of confirming or having confirmed that the subject has lung cancer based on the biopsy.

Embodiment 118. The method of embodiment 117, further comprising administering or having administered a lung cancer therapy to the subject.

Embodiment 119. The method of embodiment 118, where the lung cancer therapy is selected from the group consisting of surgery, chemotherapy, radiation therapy, targeted therapy and immunotherapy.

Embodiment 120. The method of embodiment 119, where the chemotherapy is selected from the group consisting of cisplatin or carboplatin plus docetaxel, gemcitabine, paclitaxel, vinorelbine, and pemetrexed, or a combination thereof.

Embodiment 121. The method of embodiment 119, where the targeted therapy is selected from the group consisting of Erlotinib, Afatinib, Gefitinib, Bevacizumab, Crizotinib, and Ceritinib.

Embodiment 122. A kit comprising a probe for detecting the expression level of ABCC13 for determining whether a subject is at risk of lung cancer.

Embodiment 123. The kit of embodiment 122, where the probe is a nucleic acid probe detecting the nucleic acid sequence of SEQ ID NO: 2.

Embodiment 124. The kit of embodiment 122, where the probe is a nucleic acid probe comprising nucleic acid sequence of SEQ ID NO: 1, complements thereof, or fragments thereof having at least 15 nucleotides.

Embodiment 125. The kit of embodiment 122, where the probe is an antibody, or a fragment thereof, that binds to a protein having an amino acid sequence of SEQ ID NO: 3.

Embodiment 126. The kit of embodiment 122, further comprising one or more probes for detecting the expression level of one or more biomarkers selected from the group consisting of ADCK3, GALNT14, GNAI2, PPM1M, and ZER1.

Embodiment 127. The kit of embodiment 126, where the expression level of ADCK3 is detected using a nucleic acid probe comprising nucleic acid sequence of SEQ ID NO: 4, complements thereof, or fragments thereof having at least 15 nucleotides.

Embodiment 128. The kit of embodiment 126, where the expression level of GALNT14 is detected using a nucleic acid probe comprising nucleic acid sequence of SEQ ID NO: 22, complements thereof, or fragments thereof having at least 15 nucleotides.

Embodiment 129. The kit of embodiment 126, where the expression level of GNAI2 is detected using a nucleic acid probe comprising nucleic acid sequence of SEQ ID NO: 25, complements thereof, or fragments thereof having at least 15 nucleotides.

Embodiment 130. The kit of embodiment 126, where the expression level of PPM1M is detected using a nucleic acid probe comprising nucleic acid sequence of SEQ ID NO: 91, complements thereof, or fragments thereof having at least 15 nucleotides.

Embodiment 131. The kit of embodiment 126, where the expression level of ZER1 is detected using a nucleic acid probe comprising nucleic acid sequence of SEQ ID NO: 67, complements thereof, or fragments thereof having at least 15 nucleotides.

Embodiment 132. A kit comprising a probe for detecting the expression level of ADCK3 for determining whether a subject is at risk of lung cancer.

Embodiment 133. The kit of embodiment 132, where the probe is a nucleic acid probe detecting the nucleic acid sequence of SEQ ID NO: 5.

Embodiment 134. The kit of embodiment 132, where the probe is a nucleic acid probe comprising nucleic acid sequence of SEQ ID NO: 4, complements thereof, or fragments thereof having at least 15 nucleotides.

Embodiment 135. The kit of embodiment 132, where the probe is an antibody, or a fragment thereof, that binds to a protein having an amino acid sequence of SEQ ID NO: 6.

Embodiment 136. The kit of embodiment 132, further comprising one or more probes for detecting the expression level of one or more biomarkers selected from the group consisting of ABCC13, GALNT14, GNAI2, PPM1M, and ZER1.

Embodiment 137. The kit of embodiment 136, where the expression level of ABCC13 is detected using a nucleic acid probe comprising nucleic acid sequence of SEQ ID NO: 1, complements thereof, or fragments thereof having at least 15 nucleotides.

Embodiment 138. The kit of embodiment 136, where the expression level of GALNT14 is detected using a nucleic acid probe comprising nucleic acid sequence of SEQ ID NO: 22, complements thereof, or fragments thereof having at least 15 nucleotides.

Embodiment 139. The kit of embodiment 136, where the expression level of GNAI2 is detected using a nucleic acid probe comprising nucleic acid sequence of SEQ ID NO: 25, complements thereof, or fragments thereof having at least 15 nucleotides.

Embodiment 140. The kit of embodiment 136, where the expression level of PPM1M is detected using a nucleic acid probe comprising nucleic acid sequence of SEQ ID NO: 91, complements thereof, or fragments thereof having at least 15 nucleotides.

Embodiment 141. The kit of embodiment 136, where the expression level of ZER1 is detected using a nucleic acid probe comprising nucleic acid sequence of SEQ ID NO: 67, complements thereof, or fragments thereof having at least 15 nucleotides.

Embodiment 142. A kit comprising a probe for detecting the expression level of GALNT14 for determining whether a subject is at risk of lung cancer.

Embodiment 143. The kit of embodiment 142, where the probe is a nucleic acid probe detecting the nucleic acid sequence of SEQ ID NO: 23.

Embodiment 144. The kit of embodiment 142, where the probe is a nucleic acid probe comprising nucleic acid sequence of SEQ ID NO: 22, complements thereof, or fragments thereof having at least 15 nucleotides.

Embodiment 145. The kit of embodiment 142, where the probe is an antibody, or a fragment thereof, that binds to a protein having an amino acid sequence of SEQ ID NO: 24.

Embodiment 146. The kit of embodiment 142, further comprising one or more probes for detecting the expression level of one or more biomarkers selected from the group consisting of ABCC13, ADCK3, GNAI2, PPM1M, and ZER1.

Embodiment 147. The kit of embodiment 146, where the expression level of ABCC13 is detected using a nucleic acid probe comprising nucleic acid sequence of SEQ ID NO: 1, complements thereof, or fragments thereof having at least 15 nucleotides.

Embodiment 148. The kit of embodiment 146, where the expression level of ADCK3 is detected using a nucleic acid probe comprising nucleic acid sequence of SEQ ID NO: 4, complements thereof, or fragments thereof having at least 15 nucleotides.

Embodiment 149. The kit of embodiment 146, where the expression level of GNAI2 is detected using a nucleic acid probe comprising nucleic acid sequence of SEQ ID NO: 25, complements thereof, or fragments thereof having at least 15 nucleotides.

Embodiment 150. The kit of embodiment 146, where the expression level of PPM1M is detected using a nucleic acid probe comprising nucleic acid sequence of SEQ ID NO: 91, complements thereof, or fragments thereof having at least 15 nucleotides.

Embodiment 151. The kit of embodiment 146, where the expression level of ZER1 is detected using a nucleic acid probe comprising nucleic acid sequence of SEQ ID NO: 67, complements thereof, or fragments thereof having at least 15 nucleotides.

Embodiment 152. A kit comprising a probe for detecting the expression level of GNAI2 for determining whether a subject is at risk of lung cancer.

Embodiment 153. The kit of embodiment 152, where the probe is a nucleic acid probe detecting the nucleic acid sequence of SEQ ID NO: 26.

Embodiment 154. The kit of embodiment 152, where the probe is a nucleic acid probe comprising nucleic acid sequence of SEQ ID NO: 25, complements thereof, or fragments thereof having at least 15 nucleotides.

Embodiment 155. The kit of embodiment 152, where the probe is an antibody, or a fragment thereof, that binds to a protein having an amino acid sequence of SEQ ID NO: 27.

Embodiment 156. The kit of embodiment 152, further comprising one or more probes for detecting the expression level of one or more biomarkers selected from the group consisting of ABCC13, ADCK3, GALNT14, PPM1M, and ZER1.

Embodiment 157. The kit of embodiment 156, where the expression level of ABCC13 is detected using a nucleic acid probe comprising nucleic acid sequence of SEQ ID NO: 1, complements thereof, or fragments thereof having at least 15 nucleotides.

Embodiment 158. The kit of embodiment 156, where the expression level of ADCK3 is detected using a nucleic acid probe comprising nucleic acid sequence of SEQ ID NO: 4, complements thereof, or fragments thereof having at least 15 nucleotides.

Embodiment 159. The kit of embodiment 156, where the expression level of GALNT14 is detected using a nucleic acid probe comprising nucleic acid sequence of SEQ ID NO: 22, complements thereof, or fragments thereof having at least 15 nucleotides.

Embodiment 160. The kit of embodiment 156, where the expression level of PPM1M is detected using a nucleic acid probe comprising nucleic acid sequence of SEQ ID NO: 91, complements thereof, or fragments thereof having at least 15 nucleotides.

Embodiment 161. The kit of embodiment 156, where the expression level of ZER1 is detected using a nucleic acid probe comprising nucleic acid sequence of SEQ ID NO: 67, complements thereof, or fragments thereof having at least 15 nucleotides.

Embodiment 162. A kit comprising a probe for detecting the expression level of PPM1M for determining whether a subject is at risk of lung cancer.

Embodiment 163. The kit of embodiment 162, where the probe is a nucleic acid probe detecting the nucleic acid sequence of SEQ ID NO: 92.

Embodiment 164. The kit of embodiment 162, where the probe is a nucleic acid probe comprising nucleic acid sequence of SEQ ID NO: 91, complements thereof, or fragments thereof having at least 15 nucleotides.

Embodiment 165. The kit of embodiment 162, where the probe is an antibody, or a fragment thereof, that binds to a protein having an amino acid sequence of SEQ ID NO: 93.

Embodiment 166. The kit of embodiment 162, further comprising one or more probes for detecting the expression level of one or more biomarkers selected from the group consisting of ABCC13, ADCK3, GALNT14, GNAI2, and ZER1.

Embodiment 167. The kit of embodiment 166, where the expression level of ABCC13 is detected using a nucleic acid probe comprising nucleic acid sequence of SEQ ID NO: 1, complements thereof, or fragments thereof having at least 15 nucleotides.

Embodiment 168. The kit of embodiment 166, where the expression level of ADCK3 is detected using a nucleic acid probe comprising nucleic acid sequence of SEQ ID NO: 4, complements thereof, or fragments thereof having at least 15 nucleotides.

Embodiment 169. The kit of embodiment 166, where the expression level of GALNT14 is detected using a nucleic acid probe comprising nucleic acid sequence of SEQ ID NO: 22, complements thereof, or fragments thereof having at least 15 nucleotides.

Embodiment 170. The kit of embodiment 166, where the expression level of GNAI2 is detected using a nucleic acid probe comprising nucleic acid sequence of SEQ ID NO: 25, complements thereof, or fragments thereof having at least 15 nucleotides.

Embodiment 171. The kit of embodiment 166, where the expression level of ZER1 is detected using a nucleic acid probe comprising nucleic acid sequence of SEQ ID NO: 67, complements thereof, or fragments thereof having at least 15 nucleotides.

Embodiment 172. A kit comprising a probe for detecting the expression level of ZER1 for determining whether a subject is at risk of lung cancer.

Embodiment 173. The kit of embodiment 172, where the probe is a nucleic acid probe detecting the nucleic acid sequence of SEQ ID NO: 68.

Embodiment 174. The kit of embodiment 172, where the probe is a nucleic acid probe comprising nucleic acid sequence of SEQ ID NO: 67, complements thereof, or fragments thereof having at least 15 nucleotides.

Embodiment 175. The kit of embodiment 172, where the probe is an antibody, or a fragment thereof, that binds to a protein having an amino acid sequence of SEQ ID NO: 69.

Embodiment 176. The kit of embodiment 172, further comprising one or more probes for detecting the expression level of one or more biomarkers selected from the group consisting of ABCC13, ADCK3, GALNT14, GNAI2, and PPM1M.

Embodiment 177. The kit of embodiment 176, where the expression level of ABCC13 is detected using a nucleic acid probe comprising nucleic acid sequence of SEQ ID NO: 1, complements thereof, or fragments thereof having at least 15 nucleotides.

Embodiment 178. The kit of embodiment 176, where the expression level of ADCK3 is detected using a nucleic acid probe comprising nucleic acid sequence of SEQ ID NO: 4, complements thereof, or fragments thereof having at least 15 nucleotides.

Embodiment 179. The kit of embodiment 176, where the expression level of GALNT14 is detected using a nucleic acid probe comprising nucleic acid sequence of SEQ ID NO: 22, complements thereof, or fragments thereof having at least 15 nucleotides.

Embodiment 180. The kit of embodiment 176, where the expression level of GNAI2 is detected using a nucleic acid probe comprising nucleic acid sequence of SEQ ID NO: 25, complements thereof, or fragments thereof having at least 15 nucleotides.

Embodiment 181. The kit of embodiment 176, where the expression level of PPM1M is detected using a nucleic acid probe comprising nucleic acid sequence of SEQ ID NO: 91, complements thereof, or fragments thereof having at least 15 nucleotides.

Embodiment 182. The kit of any one of embodiments 122 to 181, further comprising one or more probes for detecting the expression level of one or more biomarkers selected from the group consisting of AMD1, CLCN4, PDCD6IP, PUM1, RBPMS2, and UBAP2.

Embodiment 183. The kit of embodiment 182, where the expression level of AMD1 is detected using a nucleic acid probe comprising nucleic acid sequence of SEQ ID NO: 7, complements thereof, or fragments thereof having at least 15 nucleotides.

Embodiment 184. The kit of embodiment 182, where the expression level of CLCN4 is detected using a nucleic acid probe comprising nucleic acid sequence of SEQ ID NO: 73, complements thereof, or fragments thereof having at least 15 nucleotides.

Embodiment 185. The kit of embodiment 182, where the expression level of PDCD6IP is detected using a nucleic acid probe comprising nucleic acid sequence of SEQ ID NO: 40, complements thereof, or fragments thereof having at least 15 nucleotides.

Embodiment 186. The kit of embodiment 182, where the expression level of PUM1 is detected using a nucleic acid probe comprising nucleic acid sequence of SEQ ID NO: 94, complements thereof, or fragments thereof having at least 15 nucleotides.

Embodiment 187. The kit of embodiment 182, where the expression level of RBPMS2 is detected using a nucleic acid probe comprising nucleic acid sequence of SEQ ID NO: 97, complements thereof, or fragments thereof having at least 15 nucleotides.

Embodiment 188. The kit of embodiment 182, where the expression level of UBAP2 is detected using a nucleic acid probe comprising nucleic acid sequence of SEQ ID NO: 58, complements thereof, or fragments thereof having at least 15 nucleotides.

Embodiment 189. The kit of any one of embodiments 122 to 188, further comprising one or more probes for a set of biomarkers comprising one or more biomarkers selected from the group consisting of ANKHD1, CIR1, CLK3, CTSH, DNAJB1, EPHX2, GSR, HDAC3, IFI27, INO80C, ITPR1, LGR6, LILRA5, PNPLA6, PSMB4, SCAF4, SLC25A20, SNRPD3, STAMBP, TMEM8A, TPR, VNN1, VPS29, and WDFY3 to establish a biomarker expression profile for the subject.

Embodiment 190. The kit of embodiment 189, where the probe for ANKHD1 is a nucleic acid probe comprising nucleic acid sequence of SEQ ID NO: 70, complements thereof, or fragments thereof having at least 15 nucleotides.

Embodiment 191. The kit of embodiment 189, where the probe for CIR1 is a nucleic acid probe comprising nucleic acid sequence of SEQ ID NO: 10, complements thereof, or fragments thereof having at least 15 nucleotides.

Embodiment 192. The kit of embodiment 189, where the probe for CLK3 is a nucleic acid probe comprising nucleic acid sequence of SEQ ID NO: 13, complements thereof, or fragments thereof having at least 15 nucleotides.

Embodiment 193. The kit of embodiment 189, where the probe for CTSH is a nucleic acid probe comprising nucleic acid sequence of SEQ ID NO: 76, complements thereof, or fragments thereof having at least 15 nucleotides.

Embodiment 194. The kit of embodiment 189, where the probe for DNAJB1 is a nucleic acid probe comprising nucleic acid sequence of SEQ ID NO: 16, complements thereof, or fragments thereof having at least 15 nucleotides.

Embodiment 195. The kit of embodiment 189, where the probe for EPHX2 is a nucleic acid probe comprising nucleic acid sequence of SEQ ID NO: 19, complements thereof, or fragments thereof having at least 15 nucleotides.

Embodiment 196. The kit of embodiment 189, where the probe for GSR is a nucleic acid probe comprising nucleic acid sequence of SEQ ID NO: 28, complements thereof, or fragments thereof having at least 15 nucleotides.

Embodiment 197. The kit of embodiment 189, where the probe for HDAC3 is a nucleic acid probe comprising nucleic acid sequence of SEQ ID NO: 79, complements thereof, or fragments thereof having at least 15 nucleotides.

Embodiment 198. The kit of embodiment 189, where the probe for IFI27 is a nucleic acid probe comprising nucleic acid sequence of SEQ ID NO: 31, complements thereof, or fragments thereof having at least 15 nucleotides.

Embodiment 199. The kit of embodiment 189, where the probe for INO80C is a nucleic acid probe comprising nucleic acid sequence of SEQ ID NO: 34, complements thereof, or fragments thereof having at least 15 nucleotides.

Embodiment 200. The kit of embodiment 189, where the probe for ITPR1 is a nucleic acid probe comprising nucleic acid sequence of SEQ ID NO: 82, complements thereof, or fragments thereof having at least 15 nucleotides.

Embodiment 201. The kit of embodiment 189, where the probe for LGR6 is a nucleic acid probe comprising nucleic acid sequence of SEQ ID NO: 37, complements thereof, or fragments thereof having at least 15 nucleotides.

Embodiment 202. The kit of embodiment 189, where the probe for LILRA5 is a nucleic acid probe comprising nucleic acid sequence of SEQ ID NO: 85, complements thereof, or fragments thereof having at least 15 nucleotides.

Embodiment 203. The kit of embodiment 189, where the probe for PNPLA6 is a nucleic acid probe comprising nucleic acid sequence of SEQ ID NO: 88, complements thereof, or fragments thereof having at least 15 nucleotides.

Embodiment 204. The kit of embodiment 189, where the probe for PSMB4 is a nucleic acid probe comprising nucleic acid sequence of SEQ ID NO: 43, complements thereof, or fragments thereof having at least 15 nucleotides.

Embodiment 205. The kit of embodiment 189, where the probe for SCAF4 is a nucleic acid probe comprising nucleic acid sequence of SEQ ID NO: 46, complements thereof, or fragments thereof having at least 15 nucleotides.

Embodiment 206. The kit of embodiment 189, where the probe for SLC25A20 is a nucleic acid probe comprising nucleic acid sequence of SEQ ID NO: 100, complements thereof, or fragments thereof having at least 15 nucleotides.

Embodiment 207. The kit of embodiment 189, where the probe for SNRPD3 is a nucleic acid probe comprising nucleic acid sequence of SEQ ID NO: 49, complements thereof, or fragments thereof having at least 15 nucleotides.

Embodiment 208. The kit of embodiment 189, where the probe for STAMBP is a nucleic acid probe comprising nucleic acid sequence of SEQ ID NO: 52, complements thereof, or fragments thereof having at least 15 nucleotides.

Embodiment 209. The kit of embodiment 189, where the probe for TMEM8A is a nucleic acid probe comprising nucleic acid sequence of SEQ ID NO: 55, complements thereof, or fragments thereof having at least 15 nucleotides.

Embodiment 210. The kit of embodiment 189, where the probe for TPR is a nucleic acid probe comprising nucleic acid sequence of SEQ ID NO: 103, complements thereof, or fragments thereof having at least 15 nucleotides.

Embodiment 211. The kit of embodiment 189, where the probe for VNN1 is a nucleic acid probe comprising nucleic acid sequence of SEQ ID NO: 61, complements thereof, or fragments thereof having at least 15 nucleotides.

Embodiment 212. The kit of embodiment 189, where the probe for VPS29 is a nucleic acid probe comprising nucleic acid sequence of SEQ ID NO: 64, complements thereof, or fragments thereof having at least 15 nucleotides.

Embodiment 213. The kit of embodiment 189, where the probe for WDFY3 is a nucleic acid probe comprising nucleic acid sequence of SEQ ID NO: 106, complements thereof, or fragments thereof having at least 15 nucleotides.

Embodiment 214. The kit of embodiment 189, where the set of biomarkers comprises five or more biomarkers.

Embodiment 215. The kit of embodiment 189, where the set of biomarkers comprises ten or more biomarkers.

Embodiment 216. The kit of embodiment 189, where the set of biomarkers comprises fifteen or more biomarkers.

Embodiment 217. The kit of embodiment 189, where the set of biomarkers comprises twenty or more biomarkers.

Embodiment 218. The kit of any one of embodiments 122 to 217, where the lung cancer is non-small cell lung cancer (NSCLC).

Embodiment 219. The kit of embodiment 218, where the NSCLC is a stage I NSCLC.

Embodiment 220. The kit of embodiment 218, where the NSCLC is a stage II NSCLC.

Embodiment 221. The kit of embodiment 218, where the NSCLC is a mixture of early and late stages and types of NSCLC.

Embodiment 222. The kit of any one of embodiments 122 to 217, where the lung cancer is lung adenocarcinoma.

Embodiment 223. The kit of any one of embodiments 122 to 217, where the lung cancer is lung squamous cell carcinoma.

Embodiment 224. The kit of any one of embodiments 122 to 217, where the lung cancer is a lung carcinoid tumor.

Embodiment 225. The kit of embodiment 224, where the lung carcinoid tumor is a typical carcinoid tumor of the lung.

Embodiment 226. The kit of embodiment 224, where the lung carcinoid tumor is an atypical carcinoid tumor of the lung.

Embodiment 227. The kit of any one of embodiments 122 to 217, where the lung cancer is adenosquamous carcinoma of the lung.

While the present disclosure has been described with reference to particular embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

1. A method comprising:
determining whether a subject is at risk of lung cancer by:
obtaining or having obtained non-invasively a biological sample from the subject;
detecting expression level of ABCC13 in the biological sample; and
comparing the expression level of ABCC13 to a reference level to determine if the subject is at risk of lung cancer;
providing a recommendation that the subject is at risk of lung cancer based on said comparing.

2. A method comprising:
determining whether a subject is at risk of lung cancer by:
obtaining or having obtained non-invasively a biological sample from the subject;
detecting expression level of ABCC13 in the biological sample; and
comparing the expression level of ABCC13 to a reference level to determine if the subject is at risk of lung cancer;
if the subject is at risk of lung cancer, obtaining or having obtained a biopsy from the subject to test for lung cancer.

3. The method of claim 1 or 2, further comprising detecting expression level of a second biomarker in the sample to establish a biomarker expression profile for the subject, the second biomarker is selected from the group consisting of: ADCK3, GALNT14, GNAI2, PPM1M, and ZER1, and wherein said comparing further comprises comparing the biomarker expression profile to a reference expression profile to determine if the subject is at risk of lung cancer.

4. The method of claim 1 or 2, further comprising detecting expression level of additional biomarkers in the sample to establish a biomarker expression profile for the subject, the additional biomarkers comprise two biomarkers selected from the group consisting of: ADCK3, GALNT14, GNAI2, PPM1M, and ZER1, and wherein said comparing further comprises comparing the biomarker expression profile to a reference expression profile to determine if the subject is at risk of lung cancer.

5. The method of claim 1 or 2, further comprising detecting expression level of additional biomarkers in the sample to establish a biomarker expression profile for the subject, the additional biomarkers comprise three biomarkers selected from the group consisting of: ADCK3, GALNT14, GNAI2, PPM1M, and ZER1, and wherein said comparing further comprises comparing the biomarker expression profile to a reference expression profile to determine if the subject is at risk of lung cancer.

6. The method of claim 1 or 2, further comprising detecting expression level of additional biomarkers in the sample to establish a biomarker expression profile for the subject, the additional biomarkers comprise four biomarkers selected from the group consisting of: ADCK3, GALNT14, GNAI2, PPM1M, and ZER1, and wherein said comparing further comprises comparing the biomarker expression profile to a reference expression profile to determine if the subject is at risk of lung cancer.

7. The method of claim 1 or 2, further comprising detecting expression level of additional biomarkers in the sample to establish a biomarker expression profile for the subject, the additional biomarkers comprise ADCK3, GALNT14, GNAI2, PPM1M, and ZER1, and wherein said comparing further comprises comparing the biomarker expression profile to a reference expression profile to determine if the subject is at risk of lung cancer.

8. The method of claim 1 or 2, wherein the expression level of ABCC13 is detected using a nucleic acid probe that binds to the nucleic acid sequence of SEQ ID NO: 2.

9. The method of claim 1 or 2, wherein the expression level of ABCC13 is detected using a nucleic acid probe comprising nucleic acid sequence of SEQ ID NO: 1, complements thereof, or fragments thereof having at least 15 nucleotides.

10. The method of claim 1 or 2, wherein the expression level of ABCC13 is detected using an antibody, or a fragment thereof, that binds to a protein having an amino acid sequence of SEQ ID NO: 3.

11. A method comprising:
determining whether a subject is at risk of lung cancer by:
obtaining or having obtained non-invasively a biological sample from the subject;
detecting expression level of ADCK3 in the biological sample; and
comparing the expression level of ADCK3 to a reference level to determine if the subject is at risk of lung cancer;
providing a recommendation that the subject is at risk of lung cancer based on said comparing.

12. A method comprising:
determining whether a subject is at risk of lung cancer by:
obtaining or having obtained non-invasively a biological sample from the subject;
detecting expression level of ADCK3 in the biological sample; and
comparing the expression level of ADCK3 to a reference level to determine if the subject is at risk of lung cancer;
if the subject is at risk of lung cancer, obtaining or having obtained a biopsy from the subject to test for lung cancer.

13. The method of claim 11 or 12, further comprising detecting expression level of a second biomarker in the sample to establish a biomarker expression profile for the subject, the second biomarker is selected from the group consisting of: ABCC13, GALNT14, GNAI2, PPM1M, and ZER1, and wherein said comparing further comprises comparing the biomarker expression profile to a reference expression profile to determine if the subject is at risk of lung cancer.

14. The method of claim 11 or 12, further comprising detecting expression level of additional biomarkers in the sample to establish a biomarker expression profile for the subject, the additional biomarkers comprise two biomarkers selected from the group consisting of: ABCC13, GALNT14, GNAI2, PPM1M, and ZER1, and wherein said comparing further comprises comparing the biomarker expression profile to a reference expression profile to determine if the subject is at risk of lung cancer.

15. The method of claim 11 or 12, further comprising detecting expression level of additional biomarkers in the sample to establish a biomarker expression profile for the subject, the additional biomarkers comprise three biomarkers selected from the group consisting of: ABCC13, GALNT14, GNAI2, PPM1M, and ZER1, and wherein said comparing further comprises comparing the biomarker expression profile to a reference expression profile to determine if the subject is at risk of lung cancer.

16. The method of claim 11 or 12, further comprising detecting expression level of additional biomarkers in the sample to establish a biomarker expression profile for the subject, the additional biomarkers comprise four biomarkers selected from the group consisting of: ABCC13, GALNT14, GNAI2, PPM1M, and ZER1, and wherein said comparing further comprises comparing the biomarker expression profile to a reference expression profile to determine if the subject is at risk of lung cancer.

17. The method of claim 11 or 12, further comprising detecting expression level of additional biomarkers in the sample to establish a biomarker expression profile for the subject, the additional biomarkers comprise ABCC13, GALNT14, GNAI2, PPM1M, and ZER1, and wherein said comparing further comprises comparing the biomarker expression profile to a reference expression profile to determine if the subject is at risk of lung cancer.

18. The method of claim 11 or 12, wherein the expression level of ADCK3 is detected using a nucleic acid probe that binds to the nucleic acid sequence of SEQ ID NO: 5.

19. The method of claim 11 or 12, wherein the expression level of ADCK3 is detected using a nucleic acid probe comprising nucleic acid sequence of SEQ ID NO: 4, complements thereof, or fragments thereof having at least 15 nucleotides.

20. The method of claim 11 or 12, wherein the expression level of ADCK3 is detected using an antibody, or a fragment thereof, that binds to a protein having an amino acid sequence of SEQ ID NO: 6.

21. A method comprising:
determining whether a subject is at risk of lung cancer by:
obtaining or having obtained non-invasively a biological sample from the subject;
detecting expression level of GALNT14 in the biological sample; and
comparing the expression level of GALNT14 to a reference level to determine if the subject is at risk of lung cancer;
providing a recommendation that the subject is at risk of lung cancer based on said comparing.

22. A method comprising:
determining whether a subject is at risk of lung cancer by:
obtaining or having obtained non-invasively a biological sample from the subject;
detecting expression level of GALNT14 in the biological sample; and
comparing the expression level of GALNT14 to a reference level to determine if the subject is at risk of lung cancer;
if the subject is at risk of lung cancer, obtaining or having obtained a biopsy from the subject to test for lung cancer.

23. The method of claim 21 or 22, further comprising detecting expression level of a second biomarker in the sample to establish a biomarker expression profile for the subject, the second biomarker is selected from the group consisting of: ABCC13, ADCK3, GNAI2, PPM1M, and ZER1, and wherein said comparing further comprises comparing the biomarker expression profile to a reference expression profile to determine if the subject is at risk of lung cancer.

24. The method of claim 21 or 22, further comprising detecting expression level of additional biomarkers in the sample to establish a biomarker expression profile for the subject, the additional biomarkers comprise two biomarkers selected from the group consisting of: ABCC13, ADCK3, GNAI2, PPM1M, and ZER1, and wherein said comparing further comprises comparing the biomarker expression profile to a reference expression profile to determine if the subject is at risk of lung cancer.

25. The method of claim 21 or 22, further comprising detecting expression level of additional biomarkers in the sample to establish a biomarker expression profile for the subject, the additional biomarkers comprise three biomarkers selected from the group consisting of: ABCC13, ADCK3, GNAI2, PPM1M, and ZER1, and wherein said comparing further comprises comparing the biomarker expression profile to a reference expression profile to determine if the subject is at risk of lung cancer.

26. The method of claim 21 or 22, further comprising detecting expression level of additional biomarkers in the sample to establish a biomarker expression profile for the subject, the additional biomarkers comprise four biomarkers selected from the group consisting of: ABCC13, ADCK3, GNAI2, PPM1M, and ZER1, and wherein said comparing further comprises comparing the biomarker expression profile to a reference expression profile to determine if the subject is at risk of lung cancer.

27. The method of claim 21 or 22, further comprising detecting expression level of additional biomarkers in the sample to establish a biomarker expression profile for the subject, the additional biomarkers comprise ABCC13, ADCK3, GNAI2, PPM1M, and ZER1, and wherein said comparing further comprises comparing the biomarker expression profile to a reference expression profile to determine if the subject is at risk of lung cancer.

28. The method of claim 21 or 22, wherein the expression level of GALNT14 is detected using a nucleic acid probe that binds to the nucleic acid sequence of SEQ ID NO: 23.

29. The method of claim 21 or 22, wherein the expression level of GALNT14 is detected using a nucleic acid probe comprising nucleic acid sequence of SEQ ID NO: 22, complements thereof, or fragments thereof having at least 15 nucleotides.

30. The method of claim 21 or 22, wherein the expression level of GALNT14 is detected using an antibody, or a fragment thereof, that binds to a protein having an amino acid sequence of SEQ ID NO: 24.

31. A method comprising:
determining whether a subject is at risk of lung cancer by:
obtaining or having obtained non-invasively a biological sample from the subject;
detecting expression level of GNAI2 in the biological sample; and
comparing the expression level of GNAI2 to a reference level to determine if the subject is at risk of lung cancer;
providing a recommendation that the subject is at risk of lung cancer based on said comparing.

32. A method comprising:
determining whether a subject is at risk of lung cancer by:
obtaining or having obtained non-invasively a biological sample from the subject;
detecting expression level of GNAI2 in the biological sample; and
comparing the expression level of GNAI2 to a reference level to determine if the subject is at risk of lung cancer;
if the subject is at risk of lung cancer, obtaining or having obtained a biopsy from the subject to test for lung cancer.

33. The method of claim 31 or 32, further comprising detecting expression level of a second biomarker in the sample to establish a biomarker expression profile for the subject, the second biomarker is selected from the group consisting of: ABCC13, ADCK3, GALNT14, PPM1M, and ZER1, and wherein said comparing further comprises comparing the biomarker expression profile to a reference expression profile to determine if the subject is at risk of lung cancer.

34. The method of claim 31 or 32, further comprising detecting expression level of additional biomarkers in the sample to establish a biomarker expression profile for the subject, the additional biomarkers comprise two biomarkers selected from the group consisting of: ABCC13, ADCK3, GALNT14, PPM1M, and ZER1, and wherein said comparing further comprises comparing the biomarker expression profile to a reference expression profile to determine if the subject is at risk of lung cancer.

35. The method of claim 31 or 32, further comprising detecting expression level of additional biomarkers in the sample to establish a biomarker expression profile for the subject, the additional biomarkers comprise three biomarkers selected from the group consisting of: ABCC13, ADCK3, GALNT14, PPM1M, and ZER1, and wherein said comparing further comprises comparing the biomarker expression profile to a reference expression profile to determine if the subject is at risk of lung cancer.

36. The method of claim 31 or 32, further comprising detecting expression level of additional biomarkers in the sample to establish a biomarker expression profile for the subject, the additional biomarkers comprise four biomarkers selected from the group consisting of: ABCC13, ADCK3, GALNT14, PPM1M, and ZER1, and wherein said comparing further comprises comparing the biomarker expression profile to a reference expression profile to determine if the subject is at risk of lung cancer.

37. The method of claim 31 or 32, further comprising detecting expression level of additional biomarkers in the sample to establish a biomarker expression profile for the subject, the additional biomarkers comprise ABCC13, ADCK3, GALNT14, PPM1M, and ZER1, and wherein said comparing further comprises comparing the biomarker expression profile to a reference expression profile to determine if the subject is at risk of lung cancer.

38. The method of claim 31 or 32, wherein the expression level of GNAI2 is detected using a nucleic acid probe that binds to the nucleic acid sequence of SEQ ID NO: 26.

39. The method of claim 31 or 32, wherein the expression level of GNAI2 is detected using a nucleic acid probe comprising nucleic acid sequence of SEQ ID NO: 25, complements thereof, or fragments thereof having at least 15 nucleotides.

40. The method of claim 31 or 32, wherein the expression level of GNAI2 is detected using an antibody, or a fragment thereof, that binds to a protein having an amino acid sequence of SEQ ID NO: 27.

41. A method comprising:
determining whether a subject is at risk of lung cancer by:
obtaining or having obtained non-invasively a biological sample from the subject;
detecting expression level of PPM1M in the biological sample; and
comparing the expression level of PPM1M to a reference level to determine if the subject is at risk of lung cancer;
providing a recommendation that the subject is at risk of lung cancer based on said comparing.

42. A method comprising:
determining whether a subject is at risk of lung cancer by:
obtaining or having obtained non-invasively a biological sample from the subject;
detecting expression level of PPM1M in the biological sample; and
comparing the expression level of PPM1M to a reference level to determine if the subject is at risk of lung cancer;
if the subject is at risk of lung cancer, obtaining or having obtained a biopsy from the subject to test for lung cancer.

43. The method of claim 41 or 42, further comprising detecting expression level of a second biomarker in the sample to establish a biomarker expression profile for the subject, the second biomarker is selected from the group consisting of: ABCC13, ADCK3, GALNT14, GNAI2, and ZER1, and wherein said comparing further comprises comparing the biomarker expression profile to a reference expression profile to determine if the subject is at risk of lung cancer.

44. The method of claim 41 or 42, further comprising detecting expression level of additional biomarkers in the sample to establish a biomarker expression profile for the subject, the additional biomarkers comprise two biomarkers selected from the group consisting of: ABCC13, ADCK3, GALNT14, GNAI2, and ZER1, and wherein said comparing further comprises comparing the biomarker expression profile to a reference expression profile to determine if the subject is at risk of lung cancer.

45. The method of claim 41 or 42, further comprising detecting expression level of additional biomarkers in the sample to establish a biomarker expression profile for the subject, the additional biomarkers comprise three biomarkers selected from the group consisting of: ABCC13, ADCK3, GALNT14, GNAI2, and ZER1, and wherein said comparing further comprises comparing the biomarker expression profile to a reference expression profile to determine if the subject is at risk of lung cancer.

46. The method of claim 41 or 42, further comprising detecting expression level of additional biomarkers in the sample to establish a biomarker expression profile for the subject, the additional biomarkers comprise four biomarkers selected from the group consisting of: ABCC13, ADCK3, GALNT14, GNAI2, and ZER1, and wherein said comparing further comprises comparing the biomarker expression profile to a reference expression profile to determine if the subject is at risk of lung cancer.

47. The method of claim 41 or 42, further comprising detecting expression level of additional biomarkers in the sample to establish a biomarker expression profile for the subject, the additional biomarkers comprise ABCC13, ADCK3, GALNT14, GNAI2, and ZER1, and wherein said comparing further comprises comparing the biomarker expression profile to a reference expression profile to determine if the subject is at risk of lung cancer.

48. The method of claim 41 or 42, wherein the expression level of PPM1M is detected using a nucleic acid probe that binds to the nucleic acid sequence of SEQ ID NO: 92.

49. The method of claim 41 or 42, wherein the expression level of PPM1M is detected using a nucleic acid probe comprising nucleic acid sequence of SEQ ID NO: 91, complements thereof, or fragments thereof having at least 15 nucleotides.

50. The method of claim 41 or 42, wherein the expression level of PPM1M is detected using an antibody, or a fragment thereof, that binds to a protein having an amino acid sequence of SEQ ID NO: 93.

51. A method comprising:
determining whether a subject is at risk of lung cancer by:
obtaining or having obtained non-invasively a biological sample from the subject;
detecting expression level of ZER1 in the biological sample; and
comparing the expression level of ZER1 to a reference level to determine if the subject is at risk of lung cancer;
providing a recommendation that the subject is at risk of lung cancer based on said comparing.

52. A method comprising:
determining whether a subject is at risk of lung cancer by:
obtaining or having obtained non-invasively a biological sample from the subject;
detecting expression level of ZER1 in the biological sample; and
comparing the expression level of ZER1 to a reference level to determine if the subject is at risk of lung cancer;
if the subject is at risk of lung cancer, obtaining or having obtained a biopsy from the subject to test for lung cancer.

53. The method of claim 51 or 52, further comprising detecting expression level of a second biomarker in the sample to establish a biomarker expression profile for the subject, the second biomarker is selected from the group consisting of: ABCC13, ADCK3, GALNT14, GNAI2, and PPM1M, and wherein said comparing further comprises comparing the biomarker expression profile to a reference expression profile to determine if the subject is at risk of lung cancer.

54. The method of claim 51 or 52, further comprising detecting expression level of additional biomarkers in the sample to establish a biomarker expression profile for the subject, the additional biomarkers comprise two biomarkers selected from the group consisting of: ABCC13, ADCK3, GALNT14, GNAI2, and PPM1M, and wherein said comparing further comprises comparing the biomarker expression profile to a reference expression profile to determine if the subject is at risk of lung cancer.

55. The method of claim 51 or 52, further comprising detecting expression level of additional biomarkers in the sample to establish a biomarker expression profile for the subject, the additional biomarkers comprise three biomarkers selected from the group consisting of: ABCC13, ADCK3, GALNT14, GNAI2, and PPM1M, and wherein said comparing further comprises comparing the biomarker expression profile to a reference expression profile to determine if the subject is at risk of lung cancer.

56. The method of claim 51 or 52, further comprising detecting expression level of additional biomarkers in the sample to establish a biomarker expression profile for the subject, the additional biomarkers comprise four biomarkers selected from the group consisting of: ABCC13, ADCK3, GALNT14, GNAI2, and PPM1M, and wherein said comparing further comprises comparing the biomarker expression profile to a reference expression profile to determine if the subject is at risk of lung cancer.

57. The method of claim 51 or 52, further comprising detecting expression level of additional biomarkers in the sample to establish a biomarker expression profile for the subject, the additional biomarkers comprise ABCC13, ADCK3, GALNT14, GNAI2, and PPM1M, and wherein said comparing further comprises comparing the biomarker expression profile to a reference expression profile to determine if the subject is at risk of lung cancer.

58. The method of claim 51 or 52, wherein the expression level of ZER1 is detected using a nucleic acid probe that binds to the nucleic acid sequence of SEQ ID NO: 68.

59. The method of claim 51 or 52, wherein the expression level of ZER1 is detected using a nucleic acid probe comprising nucleic acid sequence of SEQ ID NO: 67, complements thereof, or fragments thereof having at least 15 nucleotides.

60. The method of claim 51 or 52, wherein the expression level of ZER1 is detected using an antibody, or a fragment thereof, that binds to a protein having an amino acid sequence of SEQ ID NO: 69.

61. The method of any one of claims 1 to 60, further comprising detecting expression level of one or more biomarkers selected from the group consisting of AMD1, CLCN4, PDCD6IP, PUM1, RBPMS2, and UBAP2 to establish a biomarker expression profile for the subject.

62. The method of any one of claims 1 to 60, further comprising detecting expression level of two or more biomarkers selected from the group consisting of AMD1, CLCN4, PDCD6IP, PUM1, RBPMS2, and UBAP2 to establish a biomarker expression profile for the subject.

63. The method of any one of claims 1 to 60, further comprising detecting expression level of three or more biomarkers selected from the group consisting of AMD1, CLCN4, PDCD6IP, PUM1, RBPMS2, and UBAP2 to establish a biomarker expression profile for the subject.

64. The method of any one of claims 1 to 60, further comprising detecting expression level of four or more biomarkers selected from the group consisting of AMD1, CLCN4, PDCD6IP, PUM1, RBPMS2, and UBAP2 to establish a biomarker expression profile for the subject.

65. The method of any one of claims 1 to 60, further comprising detecting expression level of five or more biomarkers selected from the group consisting of AMD1, CLCN4, PDCD6IP, PUM1, RBPMS2, and UBAP2 to establish a biomarker expression profile for the subject.

66. The method of any one of claims 1 to 60, further comprising detecting expression level of AMD1, CLCN4, PDCD6IP, PUM1, RBPMS2, and UBAP2 to establish a biomarker expression profile for the subject.

67. The method of any one of claims 61 to 66, wherein the expression level of AMD1 is detected using a nucleic acid probe comprising nucleic acid sequence of SEQ ID NO: 7, complements thereof, or fragments thereof having at least 15 nucleotides.

68. The method of any one of claims 61 to 66, wherein the expression level of CLCN4 is detected using a nucleic acid probe comprising nucleic acid sequence of SEQ ID NO: 73, complements thereof, or fragments thereof having at least 15 nucleotides.

69. The method of any one of claims 61 to 66, wherein the expression level of PDCD6IP is detected using a nucleic acid probe comprising nucleic acid sequence of SEQ ID NO: 40, complements thereof, or fragments thereof having at least 15 nucleotides.

70. The method of any one of claims 61 to 66, wherein the expression level of PUM1 is detected using a nucleic acid probe comprising nucleic acid sequence of SEQ ID NO: 94, complements thereof, or fragments thereof having at least 15 nucleotides.

71. The method of any one of claims 61 to 66, wherein the expression level of RBPMS2 is detected using a nucleic acid probe comprising nucleic acid sequence of SEQ ID NO: 97, complements thereof, or fragments thereof having at least 15 nucleotides.

72. The method of any one of claims 61 to 66, wherein the expression level of UBAP2 is detected using a nucleic acid probe comprising nucleic acid sequence of SEQ ID NO: 58, complements thereof, or fragments thereof having at least 15 nucleotides.

73. The method of any one of claims 1 to 72, further comprising detecting expression level of a set of biomarkers comprising one or more biomarkers selected from the group consisting of ANKHD1, CIR1, CLK3, CTSH, DNAJB1, EPHX2, GSR, HDAC3, IFI27, INO80C, ITPR1, LGR6, LILRA5, PNPLA6, PSMB4, SCAF4, SLC25A20, SNRPD3, STAMBP, TMEM8A, TPR, VNN1, VPS29, and WDFY3 to establish a biomarker expression profile for the subject.

74. The method of claim 73, wherein the set of biomarkers comprises five or more biomarkers.

75. The method of claim 73, wherein the set of biomarkers comprises ten or more biomarkers.

76. The method of claim 73, wherein the set of biomarkers comprises fifteen or more biomarkers.

77. The method of claim 73, wherein the set of biomarkers comprises twenty or more biomarkers.

78. The method of claim 73, wherein the set of biomarkers comprises all the biomarkers.

79. The method of any one of claims 73 to 78, wherein the expression level of ANKHD1 is detected using a nucleic acid probe comprising nucleic acid sequence of SEQ ID NO: 70, complements thereof, or fragments thereof having at least 15 nucleotides.

80. The method of any one of claims 73 to 78, wherein the expression level of CIR1 is detected using a nucleic acid probe comprising nucleic acid sequence of SEQ ID NO: 10, complements thereof, or fragments thereof having at least 15 nucleotides.

81. The method of any one of claims 73 to 78, wherein the expression level of CLK3 is detected using a nucleic acid probe comprising nucleic acid sequence of SEQ ID NO: 13, complements thereof, or fragments thereof having at least 15 nucleotides.

82. The method of any one of claims 73 to 78, wherein the expression level of CTSH is detected using a nucleic acid probe comprising nucleic acid sequence of SEQ ID NO: 76, complements thereof, or fragments thereof having at least 15 nucleotides.

83. The method of any one of claims 73 to 78, wherein the expression level of DNAJB1 is detected using a nucleic acid probe comprising nucleic acid sequence of SEQ ID NO: 16, complements thereof, or fragments thereof having at least 15 nucleotides.

84. The method of any one of claims 73 to 78, wherein the expression level of EPHX2 is detected using a nucleic acid probe comprising nucleic acid sequence of SEQ ID NO: 19, complements thereof, or fragments thereof having at least 15 nucleotides.

85. The method of any one of claims 73 to 78, wherein the expression level of GSR is detected using a nucleic acid probe comprising nucleic acid sequence of SEQ ID NO: 28, complements thereof, or fragments thereof having at least 15 nucleotides.

86. The method of any one of claims 73 to 78, wherein the expression level of HDAC3 is detected using a nucleic acid probe comprising nucleic acid sequence of SEQ ID NO: 79, complements thereof, or fragments thereof having at least 15 nucleotides.

87. The method of any one of claims 73 to 78, wherein the expression level of IFI27 is detected using a nucleic acid probe comprising nucleic acid sequence of SEQ ID NO: 31, complements thereof, or fragments thereof having at least 15 nucleotides.

88. The method of any one of claims 73 to 78, wherein the expression level of INO80C is detected using a nucleic acid probe comprising nucleic acid sequence of SEQ ID NO: 34, complements thereof, or fragments thereof having at least 15 nucleotides.

89. The method of any one of claims 73 to 78, wherein the expression level of ITPR1 is detected using a nucleic acid probe comprising nucleic acid sequence of SEQ ID NO: 82, complements thereof, or fragments thereof having at least 15 nucleotides.

90. The method of any one of claims 73 to 78, wherein the expression level of LGR6 is detected using a nucleic acid probe comprising nucleic acid sequence of SEQ ID NO: 37, complements thereof, or fragments thereof having at least 15 nucleotides.

91. The method of any one of claims 73 to 78, wherein the expression level of LILRA5 is detected using a nucleic acid probe comprising nucleic acid sequence of SEQ ID NO: 85, complements thereof, or fragments thereof having at least 15 nucleotides.

92. The method of any one of claims 73 to 78, wherein the expression level of PNPLA6 is detected using a nucleic acid probe comprising nucleic acid sequence of SEQ ID NO: 88, complements thereof, or fragments thereof having at least 15 nucleotides.

93. The method of any one of claims 73 to 78, wherein the expression level of PSMB4 is detected using a nucleic acid probe comprising nucleic acid sequence of SEQ ID NO: 43, complements thereof, or fragments thereof having at least 15 nucleotides.

94. The method of any one of claims 73 to 78, wherein the expression level of SCAF4 is detected using a nucleic acid probe comprising nucleic acid sequence of SEQ ID NO: 46, complements thereof, or fragments thereof having at least 15 nucleotides.

95. The method of any one of claims 73 to 78, wherein the expression level of SLC25A20 is detected using a nucleic acid probe comprising nucleic acid sequence of SEQ ID NO: 100, complements thereof, or fragments thereof having at least 15 nucleotides.

96. The method of any one of claims 73 to 78, wherein the expression level of SNRPD3 is detected using a nucleic acid probe comprising nucleic acid sequence of SEQ ID NO: 49, complements thereof, or fragments thereof having at least 15 nucleotides.

97. The method of any one of claims 73 to 78, wherein the expression level of STAMBP is detected using a nucleic acid probe comprising nucleic acid sequence of SEQ ID NO: 52, complements thereof, or fragments thereof having at least 15 nucleotides.

98. The method of any one of claims 73 to 78, wherein the expression level of TMEM8A is detected using a nucleic acid probe comprising nucleic acid sequence of SEQ ID NO: 55, complements thereof, or fragments thereof having at least 15 nucleotides.

99. The method of any one of claims 73 to 78, wherein the expression level of TPR is detected using a nucleic acid probe comprising nucleic acid sequence of SEQ ID NO: 103, complements thereof, or fragments thereof having at least 15 nucleotides.

100. The method of any one of claims 73 to 78, wherein the expression level of VNN1 is detected using a nucleic acid probe comprising nucleic acid sequence of SEQ ID NO: 61, complements thereof, or fragments thereof having at least 15 nucleotides.

101. The method of any one of claims 73 to 78, wherein the expression level of VPS29 is detected using a nucleic acid probe comprising nucleic acid sequence of SEQ ID NO: 64, complements thereof, or fragments thereof having at least 15 nucleotides.

102. The method of any one of claims 73 to 78, wherein the expression level of WDFY3 is detected using a nucleic acid probe comprising nucleic acid sequence of SEQ ID NO: 106, complements thereof, or fragments thereof having at least 15 nucleotides.

103. The method of any one of claims 1 to 102, wherein the biological sample is blood.

104. The method of any one of claims 1 to 103, wherein the subject is a human.

105. The method of any one of claims 1 to 104, wherein the subject has one or more lung nodules suspect of being cancerous.

106. The method of claim 105, wherein the one or more lung nodules have diameters in the range of 0.8 cm to 2 cm.

107. The method of any one of claims 1 to 106, wherein the lung cancer is non-small cell lung cancer (NSCLC).

108. The method of claim 107, wherein the NSCLC is a stage I NSCLC.

109. The method of claim 107, wherein the NSCLC is a stage II NSCLC.

110. The method of claim 107, wherein the NSCLC is a mixture of early and late stages and types of NSCLC.

111. The method of any one of claims 1 to 106, wherein the lung cancer is lung adenocarcinoma.

112. The method of any one of claims 1 to 106, wherein the lung cancer is lung squamous cell carcinoma.

113. The method of any one of claims 1 to 106, wherein the lung cancer is a lung carcinoid tumor.

114. The method of claim 113, wherein the lung carcinoid tumor is a typical carcinoid tumor of the lung.

115. The method of claim 113, wherein the lung carcinoid tumor is an atypical carcinoid tumor of the lung.

116. The method of any one of claims 1 to 106, wherein the lung cancer is adenosquamous carcinoma of the lung.

117. The method of any one of claims 2, 12, 22, 32, 42, and 52, further comprising the step of confirming or having confirmed that the subject has lung cancer based on the biopsy.

118. The method of claim 117, further comprising administering or having administered a lung cancer therapy to the subject.

119. The method of claim 118, wherein the lung cancer therapy is selected from the group consisting of surgery, chemotherapy, radiation therapy, targeted therapy and immunotherapy.

120. The method of claim 119, wherein the chemotherapy is selected from the group consisting of cisplatin or carboplatin plus docetaxel, gemcitabine, paclitaxel, vinorelbine, and pemetrexed, or a combination thereof.

121. The method of claim 119, wherein the targeted therapy is selected from the group consisting of Erlotinib, Afatinib, Gefitinib, Bevacizumab, Crizotinib, and Ceritinib.

122. A kit comprising a probe for detecting the expression level of ABCC13 for determining whether a subject is at risk of lung cancer.

123. The kit of claim 122, wherein the probe is a nucleic acid probe detecting the nucleic acid sequence of SEQ ID NO: 2.

124. The kit of claim 122, wherein the probe is a nucleic acid probe comprising nucleic acid sequence of SEQ ID NO: 1, complements thereof, or fragments thereof having at least 15 nucleotides.

125. The kit of claim 122, wherein the probe is an antibody, or a fragment thereof, that binds to a protein having an amino acid sequence of SEQ ID NO: 3.

126. The kit of claim 122, further comprising one or more probes for detecting the expression level of one or more biomarkers selected from the group consisting of ADCK3, GALNT14, GNAI2, PPM1M, and ZER1.

127. The kit of claim 126, wherein the expression level of ADCK3 is detected using a nucleic acid probe comprising nucleic acid sequence of SEQ ID NO: 4, complements thereof, or fragments thereof having at least 15 nucleotides.

128. The kit of claim 126, wherein the expression level of GALNT14 is detected using a nucleic acid probe comprising nucleic acid sequence of SEQ ID NO: 22, complements thereof, or fragments thereof having at least 15 nucleotides.

129. The kit of claim 126, wherein the expression level of GNAI2 is detected using a nucleic acid probe comprising nucleic acid sequence of SEQ ID NO: 25, complements thereof, or fragments thereof having at least 15 nucleotides.

130. The kit of claim 126, wherein the expression level of PPM1M is detected using a nucleic acid probe comprising nucleic acid sequence of SEQ ID NO: 91, complements thereof, or fragments thereof having at least 15 nucleotides.

131. The kit of claim 126, wherein the expression level of ZER1 is detected using a nucleic acid probe comprising nucleic acid sequence of SEQ ID NO: 67, complements thereof, or fragments thereof having at least 15 nucleotides.

132. A kit comprising a probe for detecting the expression level of ADCK3 for determining whether a subject is at risk of lung cancer.

133. The kit of claim 132, wherein the probe is a nucleic acid probe detecting the nucleic acid sequence of SEQ ID NO: 5.

134. The kit of claim 132, wherein the probe is a nucleic acid probe comprising nucleic acid sequence of SEQ ID NO: 4, complements thereof, or fragments thereof having at least 15 nucleotides.

135. The kit of claim 132, wherein the probe is an antibody, or a fragment thereof, that binds to a protein having an amino acid sequence of SEQ ID NO: 6.

136. The kit of claim 132, further comprising one or more probes for detecting the expression level of one or more biomarkers selected from the group consisting of ABCC13, GALNT14, GNAI2, PPM1M, and ZER1.

137. The kit of claim 136, wherein the expression level of ABCC13 is detected using a nucleic acid probe comprising nucleic acid sequence of SEQ ID NO: 1, complements thereof, or fragments thereof having at least 15 nucleotides.

138. The kit of claim 136, wherein the expression level of GALNT14 is detected using a nucleic acid probe comprising nucleic acid sequence of SEQ ID NO: 22, complements thereof, or fragments thereof having at least 15 nucleotides.

139. The kit of claim 136, wherein the expression level of GNAI2 is detected using a nucleic acid probe comprising nucleic acid sequence of SEQ ID NO: 25, complements thereof, or fragments thereof having at least 15 nucleotides.

140. The kit of claim 136, wherein the expression level of PPM1M is detected using a nucleic acid probe comprising nucleic acid sequence of SEQ ID NO: 91, complements thereof, or fragments thereof having at least 15 nucleotides.

141. The kit of claim 136, wherein the expression level of ZER1 is detected using a nucleic acid probe comprising nucleic acid sequence of SEQ ID NO: 67, complements thereof, or fragments thereof having at least 15 nucleotides.

142. A kit comprising a probe for detecting the expression level of GALNT14 for determining whether a subject is at risk of lung cancer.

143. The kit of claim 142, wherein the probe is a nucleic acid probe detecting the nucleic acid sequence of SEQ ID NO: 23.

144. The kit of claim 142, wherein the probe is a nucleic acid probe comprising nucleic acid sequence of SEQ ID NO: 22, complements thereof, or fragments thereof having at least 15 nucleotides.

145. The kit of claim 142, wherein the probe is an antibody, or a fragment thereof, that binds to a protein having an amino acid sequence of SEQ ID NO: 24.

146. The kit of claim 142, further comprising one or more probes for detecting the expression level of one or more biomarkers selected from the group consisting of ABCC13, ADCK3, GNAI2, PPM1M, and ZER1.

147. The kit of claim 146, wherein the expression level of ABCC13 is detected using a nucleic acid probe comprising nucleic acid sequence of SEQ ID NO: 1, complements thereof, or fragments thereof having at least 15 nucleotides.

148. The kit of claim 146, wherein the expression level of ADCK3 is detected using a nucleic acid probe comprising nucleic acid sequence of SEQ ID NO: 4, complements thereof, or fragments thereof having at least 15 nucleotides.

149. The kit of claim 146, wherein the expression level of GNAI2 is detected using a nucleic acid probe comprising nucleic acid sequence of SEQ ID NO: 25, complements thereof, or fragments thereof having at least 15 nucleotides.

150. The kit of claim 146, wherein the expression level of PPM1M is detected using a nucleic acid probe comprising nucleic acid sequence of SEQ ID NO: 91, complements thereof, or fragments thereof having at least 15 nucleotides.

151. The kit of claim 146, wherein the expression level of ZER1 is detected using a nucleic acid probe comprising nucleic acid sequence of SEQ ID NO: 67, complements thereof, or fragments thereof having at least 15 nucleotides.

152. A kit comprising a probe for detecting the expression level of GNAI2 for determining whether a subject is at risk of lung cancer.

153. The kit of claim 152, wherein the probe is a nucleic acid probe detecting the nucleic acid sequence of SEQ ID NO: 26.

154. The kit of claim 152, wherein the probe is a nucleic acid probe comprising nucleic acid sequence of SEQ ID NO: 25, complements thereof, or fragments thereof having at least 15 nucleotides.

155. The kit of claim 152, wherein the probe is an antibody, or a fragment thereof, that binds to a protein having an amino acid sequence of SEQ ID NO: 27.

156. The kit of claim 152, further comprising one or more probes for detecting the expression level of one or more biomarkers selected from the group consisting of ABCC13, ADCK3, GALNT14, PPM1M, and ZER1.

157. The kit of claim 156, wherein the expression level of ABCC13 is detected using a nucleic acid probe comprising nucleic acid sequence of SEQ ID NO: 1, complements thereof, or fragments thereof having at least 15 nucleotides.

158. The kit of claim 156, wherein the expression level of ADCK3 is detected using a nucleic acid probe comprising nucleic acid sequence of SEQ ID NO: 4, complements thereof, or fragments thereof having at least 15 nucleotides.

159. The kit of claim 156, wherein the expression level of GALNT14 is detected using a nucleic acid probe comprising nucleic acid sequence of SEQ ID NO: 22, complements thereof, or fragments thereof having at least 15 nucleotides.

160. The kit of claim 156, wherein the expression level of PPM1M is detected using a nucleic acid probe comprising nucleic acid sequence of SEQ ID NO: 91, complements thereof, or fragments thereof having at least 15 nucleotides.

161. The kit of claim 156, wherein the expression level of ZER1 is detected using a nucleic acid probe comprising nucleic acid sequence of SEQ ID NO: 67, complements thereof, or fragments thereof having at least 15 nucleotides.

162. A kit comprising a probe for detecting the expression level of PPM1M for determining whether a subject is at risk of lung cancer.

163. The kit of claim 162, wherein the probe is a nucleic acid probe detecting the nucleic acid sequence of SEQ ID NO: 92.

164. The kit of claim 162, wherein the probe is a nucleic acid probe comprising nucleic acid sequence of SEQ ID NO: 91, complements thereof, or fragments thereof having at least 15 nucleotides.

165. The kit of claim 162, wherein the probe is an antibody, or a fragment thereof, that binds to a protein having an amino acid sequence of SEQ ID NO: 93.

166. The kit of claim 162, further comprising one or more probes for detecting the expression level of one or more biomarkers selected from the group consisting of ABCC13, ADCK3, GALNT14, GNAI2, and ZER1.

167. The kit of claim 166, wherein the expression level of ABCC13 is detected using a nucleic acid probe comprising nucleic acid sequence of SEQ ID NO: 1, complements thereof, or fragments thereof having at least 15 nucleotides.

168. The kit of claim 166, wherein the expression level of ADCK3 is detected using a nucleic acid probe comprising nucleic acid sequence of SEQ ID NO: 4, complements thereof, or fragments thereof having at least 15 nucleotides.

169. The kit of claim 166, wherein the expression level of GALNT14 is detected using a nucleic acid probe comprising nucleic acid sequence of SEQ ID NO: 22, complements thereof, or fragments thereof having at least 15 nucleotides.

170. The kit of claim 166, wherein the expression level of GNAI2 is detected using a nucleic acid probe comprising nucleic acid sequence of SEQ ID NO: 25, complements thereof, or fragments thereof having at least 15 nucleotides.

171. The kit of claim 166, wherein the expression level of ZER1 is detected using a nucleic acid probe comprising nucleic acid sequence of SEQ ID NO: 67, complements thereof, or fragments thereof having at least 15 nucleotides.

172. A kit comprising a probe for detecting the expression level of ZER1 for determining whether a subject is at risk of lung cancer.

173. The kit of claim 172, wherein the probe is a nucleic acid probe detecting the nucleic acid sequence of SEQ ID NO: 68.

174. The kit of claim 172, wherein the probe is a nucleic acid probe comprising nucleic acid sequence of SEQ ID NO: 67, complements thereof, or fragments thereof having at least 15 nucleotides.

175. The kit of claim 172, wherein the probe is an antibody, or a fragment thereof, that binds to a protein having an amino acid sequence of SEQ ID NO: 69.

176. The kit of claim 172, further comprising one or more probes for detecting the expression level of one or more biomarkers selected from the group consisting of ABCC13, ADCK3, GALNT14, GNAI2, and PPM1M.

177. The kit of claim 176, wherein the expression level of ABCC13 is detected using a nucleic acid probe comprising nucleic acid sequence of SEQ ID NO: 1, complements thereof, or fragments thereof having at least 15 nucleotides.

178. The kit of claim 176, wherein the expression level of ADCK3 is detected using a nucleic acid probe comprising nucleic acid sequence of SEQ ID NO: 4, complements thereof, or fragments thereof having at least 15 nucleotides.

179. The kit of claim 176, wherein the expression level of GALNT14 is detected using a nucleic acid probe comprising nucleic acid sequence of SEQ ID NO: 22, complements thereof, or fragments thereof having at least 15 nucleotides.

180. The kit of claim 176, wherein the expression level of GNAI2 is detected using a nucleic acid probe comprising nucleic acid sequence of SEQ ID NO: 25, complements thereof, or fragments thereof having at least 15 nucleotides.

181. The kit of claim 176, wherein the expression level of PPM1M is detected using a nucleic acid probe comprising nucleic acid sequence of SEQ ID NO: 91, complements thereof, or fragments thereof having at least 15 nucleotides.

182. The kit of any one of claims 122 to 181, further comprising one or more probes for detecting the expression level of one or more biomarkers selected from the group consisting of AMD1, CLCN4, PDCD6IP, PUM1, RBPMS2, and UBAP2.

183. The kit of claim 182, wherein the expression level of AMD1 is detected using a nucleic acid probe comprising nucleic acid sequence of SEQ ID NO: 7, complements thereof, or fragments thereof having at least 15 nucleotides.

184. The kit of claim 182, wherein the expression level of CLCN4 is detected using a nucleic acid probe comprising nucleic acid sequence of SEQ ID NO: 73, complements thereof, or fragments thereof having at least 15 nucleotides.

185. The kit of claim 182, wherein the expression level of PDCD6IP is detected using a nucleic acid probe comprising nucleic acid sequence of SEQ ID NO: 40, complements thereof, or fragments thereof having at least 15 nucleotides.

186. The kit of claim 182, wherein the expression level of PUM1 is detected using a nucleic acid probe comprising nucleic acid sequence of SEQ ID NO: 94, complements thereof, or fragments thereof having at least 15 nucleotides.

187. The kit of claim 182, wherein the expression level of RBPMS2 is detected using a nucleic acid probe comprising nucleic acid sequence of SEQ ID NO: 97, complements thereof, or fragments thereof having at least 15 nucleotides.

188. The kit of claim 182, wherein the expression level of UBAP2 is detected using a nucleic acid probe comprising nucleic acid sequence of SEQ ID NO: 58, complements thereof, or fragments thereof having at least 15 nucleotides.

189. The kit of any one of claims 122 to 188, further comprising one or more probes for a set of biomarkers comprising one or more biomarkers selected from the group consisting of ANKHD1, CIR1, CLK3, CTSH, DNAJB1, EPHX2, GSR, HDAC3, IFI27, INO80C, ITPR1, LGR6, LILRA5, PNPLA6, PSMB4, SCAF4, SLC25A20, SNRPD3, STAMBP, TMEM8A, TPR, VNN1, VPS29, and WDFY3 to establish a biomarker expression profile for the subject.

190. The kit of claim 189, wherein the probe for ANKHD1 is a nucleic acid probe comprising nucleic acid sequence of SEQ ID NO: 70, complements thereof, or fragments thereof having at least 15 nucleotides.

191. The kit of claim 189, wherein the probe for CIR1 is a nucleic acid probe comprising nucleic acid sequence of SEQ ID NO: 10, complements thereof, or fragments thereof having at least 15 nucleotides.

192. The kit of claim 189, wherein the probe for CLK3 is a nucleic acid probe comprising nucleic acid sequence of SEQ ID NO: 13, complements thereof, or fragments thereof having at least 15 nucleotides.

193. The kit of claim 189, wherein the probe for CTSH is a nucleic acid probe comprising nucleic acid sequence of SEQ ID NO: 76, complements thereof, or fragments thereof having at least 15 nucleotides.

194. The kit of claim 189, wherein the probe for DNAJB1 is a nucleic acid probe comprising nucleic acid sequence of SEQ ID NO: 16, complements thereof, or fragments thereof having at least 15 nucleotides.

195. The kit of claim 189, wherein the probe for EPHX2 is a nucleic acid probe comprising nucleic acid sequence of SEQ ID NO: 19, complements thereof, or fragments thereof having at least 15 nucleotides.

196. The kit of claim 189, wherein the probe for GSR is a nucleic acid probe comprising nucleic acid sequence of SEQ ID NO: 28, complements thereof, or fragments thereof having at least 15 nucleotides.

197. The kit of claim 189, wherein the probe for HDAC3 is a nucleic acid probe comprising nucleic acid sequence of SEQ ID NO: 79, complements thereof, or fragments thereof having at least 15 nucleotides.

198. The kit of claim 189, wherein the probe for IFI27 is a nucleic acid probe comprising nucleic acid sequence of SEQ ID NO: 31, complements thereof, or fragments thereof having at least 15 nucleotides.

199. The kit of claim 189, wherein the probe for INO80C is a nucleic acid probe comprising nucleic acid sequence of SEQ ID NO: 34, complements thereof, or fragments thereof having at least 15 nucleotides.

200. The kit of claim 189, wherein the probe for ITPR1 is a nucleic acid probe comprising nucleic acid sequence of SEQ ID NO: 82, complements thereof, or fragments thereof having at least 15 nucleotides.

201. The kit of claim 189, wherein the probe for LGR6 is a nucleic acid probe comprising nucleic acid sequence of SEQ ID NO: 37, complements thereof, or fragments thereof having at least 15 nucleotides.

202. The kit of claim 189, wherein the probe for LILRA5 is a nucleic acid probe comprising nucleic acid sequence of SEQ ID NO: 85, complements thereof, or fragments thereof having at least 15 nucleotides.

203. The kit of claim 189, wherein the probe for PNPLA6 is a nucleic acid probe comprising nucleic acid sequence of SEQ ID NO: 88, complements thereof, or fragments thereof having at least 15 nucleotides.

204. The kit of claim 189, wherein the probe for PSMB4 is a nucleic acid probe comprising nucleic acid sequence of SEQ ID NO: 43, complements thereof, or fragments thereof having at least 15 nucleotides.

205. The kit of claim 189, wherein the probe for SCAF4 is a nucleic acid probe comprising nucleic acid sequence of SEQ ID NO: 46, complements thereof, or fragments thereof having at least 15 nucleotides.

206. The kit of claim 189, wherein the probe for SLC25A20 is a nucleic acid probe comprising nucleic acid sequence of SEQ ID NO: 100, complements thereof, or fragments thereof having at least 15 nucleotides.

207. The kit of claim 189, wherein the probe for SNRPD3 is a nucleic acid probe comprising nucleic acid sequence of SEQ ID NO: 49, complements thereof, or fragments thereof having at least 15 nucleotides.

208. The kit of claim 189, wherein the probe for STAMBP is a nucleic acid probe comprising nucleic acid sequence of SEQ ID NO: 52, complements thereof, or fragments thereof having at least 15 nucleotides.

209. The kit of claim 189, wherein the probe for TMEM8A is a nucleic acid probe comprising nucleic acid sequence of SEQ ID NO: 55, complements thereof, or fragments thereof having at least 15 nucleotides.

210. The kit of claim 189, wherein the probe for TPR is a nucleic acid probe comprising nucleic acid sequence of SEQ ID NO: 103, complements thereof, or fragments thereof having at least 15 nucleotides.

211. The kit of claim 189, wherein the probe for VNN1 is a nucleic acid probe comprising nucleic acid sequence of SEQ ID NO: 61, complements thereof, or fragments thereof having at least 15 nucleotides.

212. The kit of claim 189, wherein the probe for VPS29 is a nucleic acid probe comprising nucleic acid sequence of SEQ ID NO: 64, complements thereof, or fragments thereof having at least 15 nucleotides.

213. The kit of claim 189, wherein the probe for WDFY3 is a nucleic acid probe comprising nucleic acid sequence of SEQ ID NO: 106, complements thereof, or fragments thereof having at least 15 nucleotides.

214. The kit of claim 189, wherein the set of biomarkers comprises five or more biomarkers.

215. The kit of claim 189, wherein the set of biomarkers comprises ten or more biomarkers.

216. The kit of claim 189, wherein the set of biomarkers comprises fifteen or more biomarkers.

217. The kit of claim 189, wherein the set of biomarkers comprises twenty or more biomarkers.

218. The kit of any one of claims 122 to 217, wherein the lung cancer is non-small cell lung cancer (NSCLC).

219. The kit of claim 218, wherein the NSCLC is a stage I NSCLC.

220. The kit of claim 218, wherein the NSCLC is a stage II NSCLC.

221. The kit of claim 218, wherein the NSCLC is a mixture of early and late stages and types of NSCLC.

222. The kit of any one of claims 122 to 217, wherein the lung cancer is lung adenocarcinoma.

223. The kit of any one of claims 122 to 217, wherein the lung cancer is lung squamous cell carcinoma.

224. The kit of any one of claims 122 to 217, wherein the lung cancer is a lung carcinoid tumor.

225. The kit of claim 224, wherein the lung carcinoid tumor is a typical carcinoid tumor of the lung.

226. The kit of claim 224, wherein the lung carcinoid tumor is an atypical carcinoid tumor of the lung.

227. The kit of any one of claims 122 to 217, wherein the lung cancer is adenosquamous carcinoma of the lung.
